(19) United States
(12) Patent Application Publication
(10) Pub. No.: US 2019/0343782 A1
(43) Pub. Date: Nov. 14, 2019

(54) METHODS OF TREATING LIVER TOXICITY AND DISORDERS

(71) Applicant: Yeda Research and Development Co. Ltd., Rehovot (IL)

(72) Inventors: Eran ELINAV, Mazkeret Batia (IL); Eran SEGAL, Ramat-HaSharon (IL)

(73) Assignee: Yeda Research and Development Co. Ltd., Rehovot (IL)

(21) Appl. No.: 16/465,149

(22) PCT Filed: Nov. 28, 2017
(86) PCT No.: PCT/IL2017/051297

§ 371 Date: May 30, 2019

Related U.S. Application Data
(60) Provisional application No. 62/451,778, filed on Jan. 30, 2017.
(60) Provisional application No. 62/427,947, filed on Nov. 30, 2016.

(51) Int. Cl. A61K 31/167; A61P 1/16; A61K 45/06

(52) U.S. Cl. CPC A61K 31/167; A61K 45/06; A61P 1/16

(57) ABSTRACT

A method of reducing the liver toxicity of a liver-damaging agent is disclosed. The method comprises administering to the subject:
(i) the liver-damaging agent; and
(ii) an agent which alters the circadian rhythmicity of microbes of the gut microbiome of the subject.

### FIELD AND BACKGROUND OF THE INVENTION

The present invention, in some embodiments thereof, relates to methods of treating liver toxicity and disorders. The methods rely on altering metabolite rhythms and programming of transcriptional oscillations of the liver via manipulation of the circadian activity of the host's gut microbiome.

The human intestine carries a vast and diverse microbial ecosystem that has co-evolved with our species and is essential for human health. Mammals possess an ‘extended genome’ of millions of microbial genes located in the intestine: the microbiome. This multigenomic symbiosis is expressed at the proteomic and metabolic levels in the host and it has therefore been proposed that humans represent a vastly complex biological ‘superorganism’ in which part of the responsibility for host metabolic regulation is devolved to the microbial symbionts. Modern interpretation of the gut microbiome is based on a culture-independent, molecular view of the intestine provided by high-throughput genomic screening technologies. Also, the gut microbiome has been directly implicated in the etiopathogenesis of a number of pathological states as diverse as obesity, circulatory disease, inflammatory bowel diseases (IBDs) and autism. The gut microbiota also influences drug metabolism and toxicity, dietary calorific bioavailability, immune system conditioning and response, and post-surgical recovery. The implication is that quantitative analysis of the gut microbiome and its activities is essential for the generation of future personalized healthcare strategies and that the gut microbiome represents a fertile ground for the development of the next generation of therapeutic drug targets. It also implies that the gut microbiome may be directly modulated for the benefit of the host organism.

The gut microbiota therefore perform a large number of important roles that define the physiology of the host, such as immune system maturation, the intestinal response to epithelial cell injury, and xenobiotic and energy metabolism.

The mammalian circadian clock adjusts physiological processes to diurnal environmental variations through the coordination of transcriptome oscillations in peripheral tissues. In each individual cell, the rhythmic transcriptional program is carried out by a network of core clock transcription factors, including period (Per), cryptochrome (Cry), Bmal (Arntl), and Clock, with nuclear receptors of the ROR and REV-ERB families stabilizing the core oscillator. These factors control rhythmic chromatin dynamics, including rhythmic changes in epigenetic marks at circadian promoters, spatial chromosome arrangement, and polymerase activity, thereby determining the fraction of the genome undergoing oscillating expression in a tissue-specific manner. As a result, up to 20 percent of a tissue's total transcriptome and up to 50 percent of all transcripts in the body consist of oscillating elements, which determine the diurnal pattern of cellular and organismal activity.

Cellular metabolism is greatly affected by the activity of the circadian clock, thereby accommodating the temporal variation of an organism's metabolic needs over the course of a day. For instance, the rhythmic fluctuations of metabolite levels in a given tissue are driven by the components of the molecular clock. In turn, metabolic products serve as an important input into the circadian clock network, thereby creating a bi-directional feedback loop of reciprocal control between cellular metabolic activity and the circadian clock.

In addition to the circadian variation in the physiology of the host, it was recently discovered that the gut microbiota undergoes diurnal oscillations in composition and function. These oscillations are controlled by the timing of food intake and the composition of the diet. The diurnal interaction between the host and its gut microbiome can affect circadian clock activity in different tissues and is particularly critical for metabolic homeostasis of the host, as failure to rhythmically control the microbiota results in dysbiosis that promotes obesity and other manifestations of the metabolic syndrome. However, how the rhythmic activity of the microbiota feeds into the circadian clock network both locally in the intestine and systemically, and how this concerted host-microbiome oscillation impacts diurnal organ physiology remains elusive.

Additional background art includes WO2015/166492 and Clayton et al., Proc Natl Acad Sci USA. 2009 Aug. 25; 106(34): 14728-14733.

### SUMMARY OF THE INVENTION

According to an aspect of the present invention there is provided a method of reducing the liver toxicity of a liver-damaging agent in a subject comprising administering to the subject:

(i) the liver-damaging agent; and

(ii) an agent which alters the circadian rhythmicity of microbes of the gut microbiome of the subject, thereby reducing the liver toxicity of the liver-damaging agent.

According to an aspect of the present invention there is provided a method of treating a liver disease in a subject in need thereof comprising administering to the subject:

(i) a therapeutically effective amount of an agent which is therapeutic for the liver disease; and

(ii) an agent which alters the circadian rhythmicity of microbes of the gut microbiome of the subject, thereby treating the liver disease.

According to an aspect of the present invention there is provided a combination of an agent which alters the circadian rhythmicity of microbes of the gut microbiome of a subject and a liver-damaging agent for reducing the liver toxicity of the liver-damaging agent.

According to an aspect of the present invention there is provided a combination of an agent which is therapeutic for a liver disease and an agent which alters the circadian rhythmicity of microbes of the gut microbiome of a subject for treating a liver disease.

According to an aspect of the present invention there is provided a method of selecting a dose or treatment regimen of a pharmaceutical agent for a subject comprising:

(a) analyzing the circadian rhythmicity of at least one component of the gut microbiome of the subject; and

(b) selecting the dose or treatment regimen according to the circadian rhythmicity. According to embodiments of the present invention, the agent which alters the circadian rhythmicity of microbes of the gut microbiome is an antibiotic.

According to embodiments of the present invention, the antibiotic is selected from the group consisting of Ampicillin, Neomycin, Vancomycin and Mettronidazole.

According to embodiments of the present invention, the agent which alters the circadian rhythmicity of microbes of the gut microbiome is a probiotic.

According to embodiments of the present invention, the probiotic comprises a polyamine producing bacteria.

According to embodiments of the present invention, the agent comprises a food which is rich in polyamines.

According to embodiments of the present invention, the agent comprises a diet which is a polyamine deficient diet or a fat rich diet.

According to embodiments of the present invention, the agent is a metabolite whose abundance follows a circadian rhythmicity in the gut microbiome.

According to embodiments of the present invention, the at least one component is a metabolite.

According to embodiments of the present invention, the metabolite is a polyamine.

According to embodiments of the present invention, the polyamine is selected from the group consisting of putrescine, cadaverine, spermidine and spermine.

According to embodiments of the present invention, the agent is a fecal microbiome transplant derived from a healthy donor.

According to embodiments of the present invention, the liver-damaging agent is a pharmaceutical agent.

According to embodiments of the present invention, the pharmaceutical agent is acetaminophen.

According to embodiments of the present invention, the liver-damaging agent is a diagnostic agent.

According to embodiments of the present invention, the liver-damaging agent is administered in a higher unit dosage than the pharmaceutically acceptable unit dosage thereof.

According to embodiments of the present invention, the therapeutically effective amount of the agent is higher than the pharmaceutically acceptable unit dosage of the agent.

According to embodiments of the present invention, the agent which alters the circadian rhythmicity of the gut microbiome is selected by analyzing the gut microbiome of the subject prior to the administering.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-I. Mucosal proximity of the intestinal microbiota undergoes diurnal fluctuations.

(A) Schematic showing sampling times for luminal and mucosal-adherent communities.

(B) SEM images showing diurnal fluctuations in epithelial colonization by bacteria. Images are representative of 10 randomly chosen views per mouse.

(C) Diurnal fluctuations in the number of bacteria attached to colonic epithelium over two dark-light cycles as determined by bacterial qPCR of adherent communities.

(D) Relative taxonomic composition of mucosal-adherent bacteria over the course of two light-dark cycles.

(E) Principal coordinate analysis (PCoA) of mucosal-adherent bacteria over the course of two light-dark cycles.

(F) Epithelial-adherent operational taxonomic units (OTUs) showing diurnal oscillations in absolute abundance. Fluctuation amplitudes are depicted. Dashed line indicates q<0.1.

(G) Example of a bacterial species showing fluctuating absolute numbers in mucosal-adherent communities.

(H, I) Dot plot (H) and heatmap (I) of diurnal gene abundance oscillations in mucosal commensal bacteria. Genes with p<0.05 and q<0.1 are shown.

Data are representative of two independent experiments with N=45 (A-G) or N=18 mice (H, I) mice. JTK_cycle was used to calculate p- and q-values.

FIGS. 2A-I. Bacterial motility and mucus degradation undergoes diurnal fluctuations.

(A) KEGG pathways of mucosal-associated microbial communities showing diurnal oscillations in relative abundance.

(B) Diurnal fluctuations in the relative abundance of bacterial chemotaxis in mucosal-associated communities.

(C, D) Quantification (C) and representative immunofluorescence images (D) showing diurnal fluctuations in epithelial proximity by commensal bacteria. Stained are Muc2 protein (green) and bacterial 16S rDNA (red). Images are representative of 10 randomly chosen views per mouse. Arrows indicate bacterial invasion into the mucus layer. Scale bars indicate 100 μm.

(E) Comparative immunofluorescence images showing epithelial proximity of commensal bacteria in wild-type and Reg3g^{-/-} mice at ZT12.

(F) Fluctuations in the number of bacteria attached to intestinal epithelium in wild-type and Reg3g^{-/-} mice over two dark-light cycles as determined by bacterial qPCR of adherent communities.

(G, H) Numbers of mucosal-resident bacteria in Per1/2^{-/-} mice that were either fed ad libitum (G) or only during the light phase (H).

(I) Epithelial-adherent operational taxonomic units (OTUs) showing diurnal oscillations in relative abundance in Per1/2^{-/-} mice that were either fed ad libitum or only during the light phase.

Data are representative of 1-2 independent experiments with N=18-45 mice. JTK_cycle was used to calculate p- and q-values.

FIGS. 3A-L. Antibiotic treatment abrogates microbial adherence rhythms and reprograms intestinal transcriptome oscillations.

(A) Schematic showing sampling times for colonic transcriptome, epigenome, and metabolome in antibiotics-treated mice and controls.

(B) Diurnal fluctuations in the number of bacteria attached to colonic epithelium over the course of two dark-light cycles in antibiotics-treated mice and controls.

(C) Representative SEM images showing epithelial colonization by bacteria at ZTO in antibiotics-treated mice and controls.

(D) Epithelial-adherent OTUs showing diurnal oscillations in relative abundance in antibiotics-treated mice and controls.

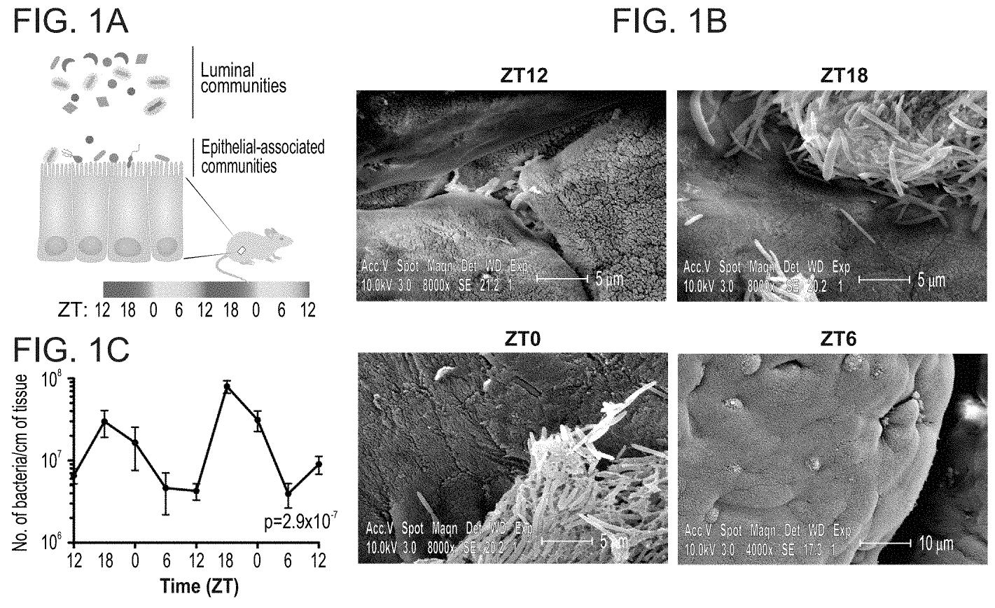

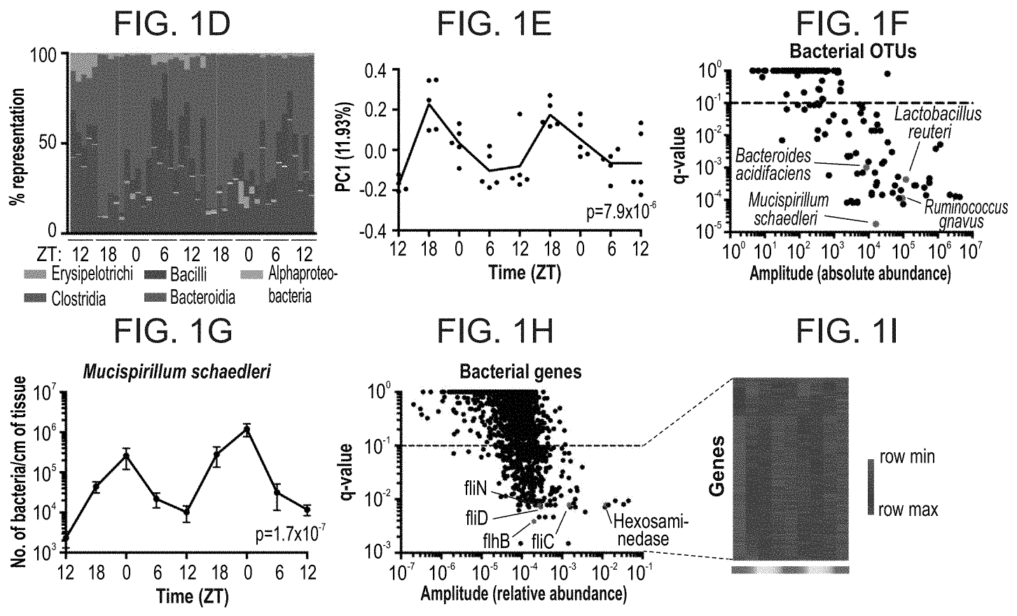

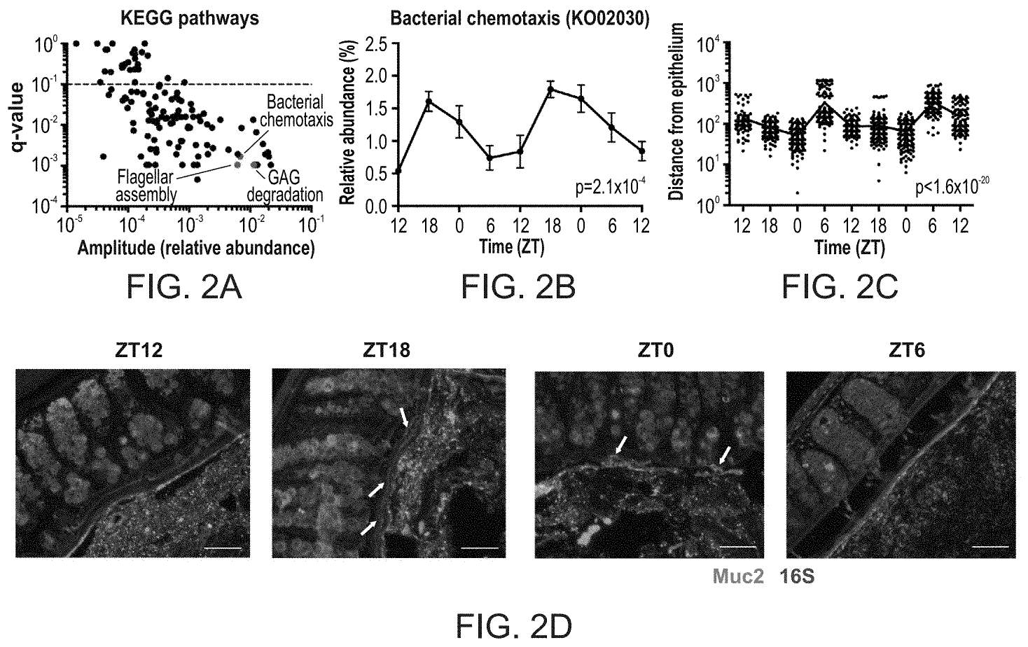

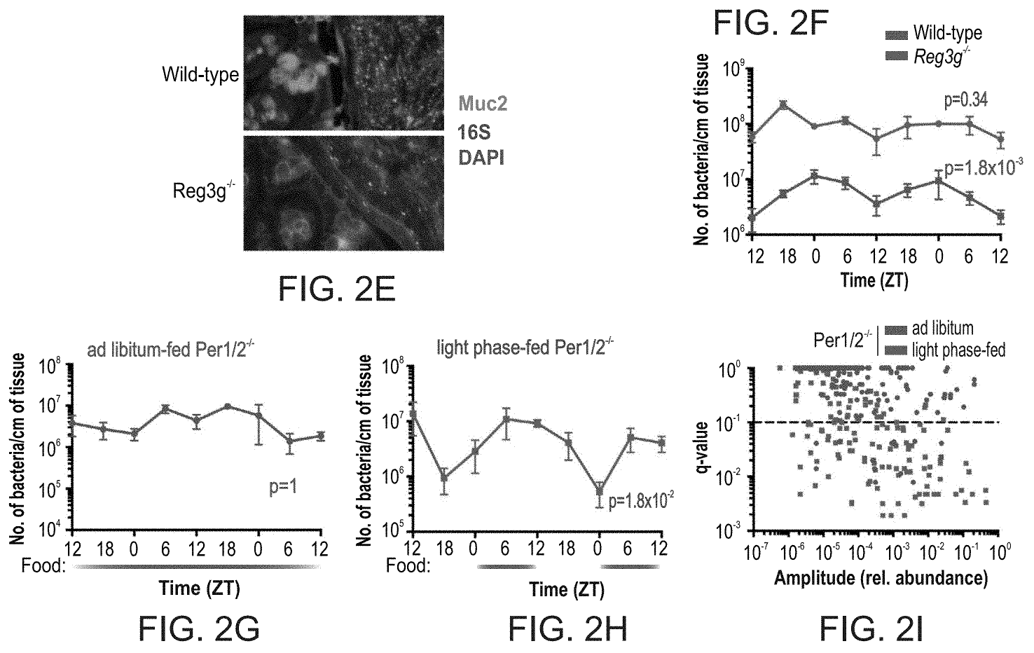

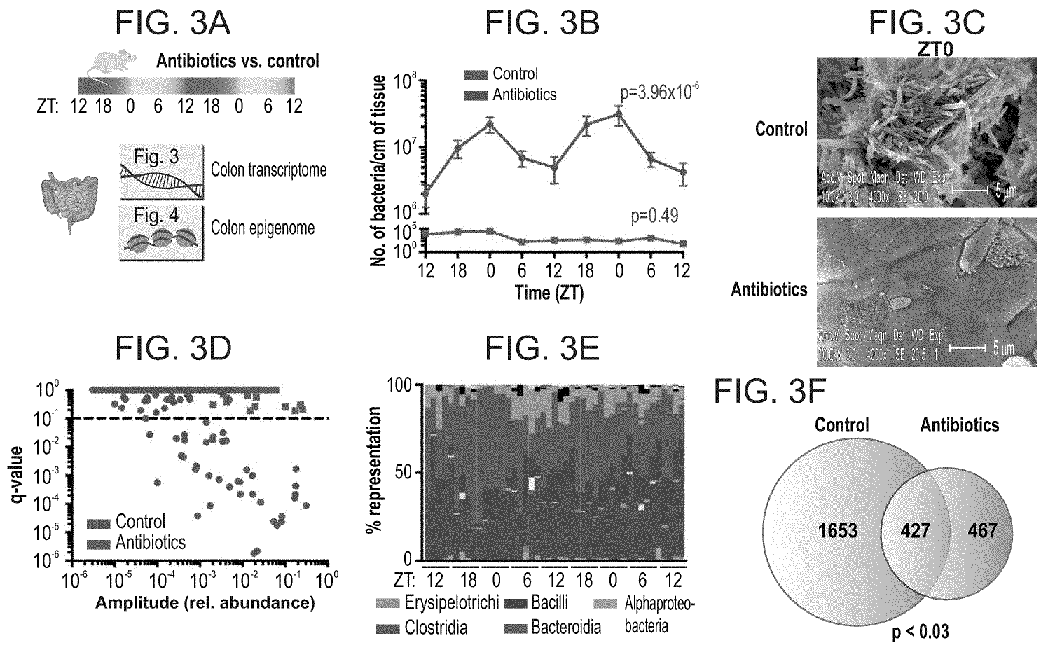

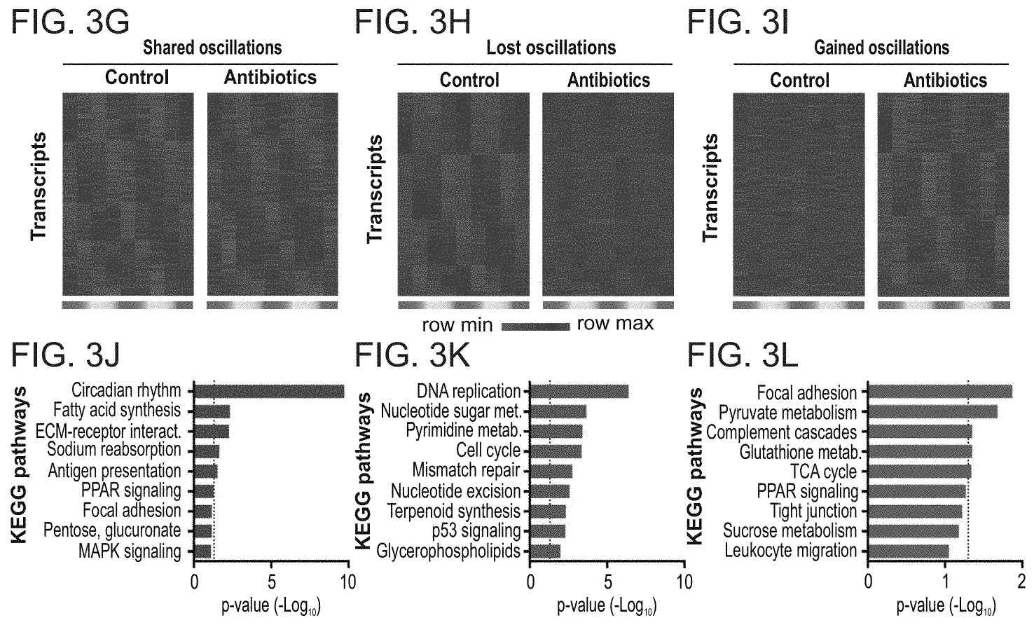

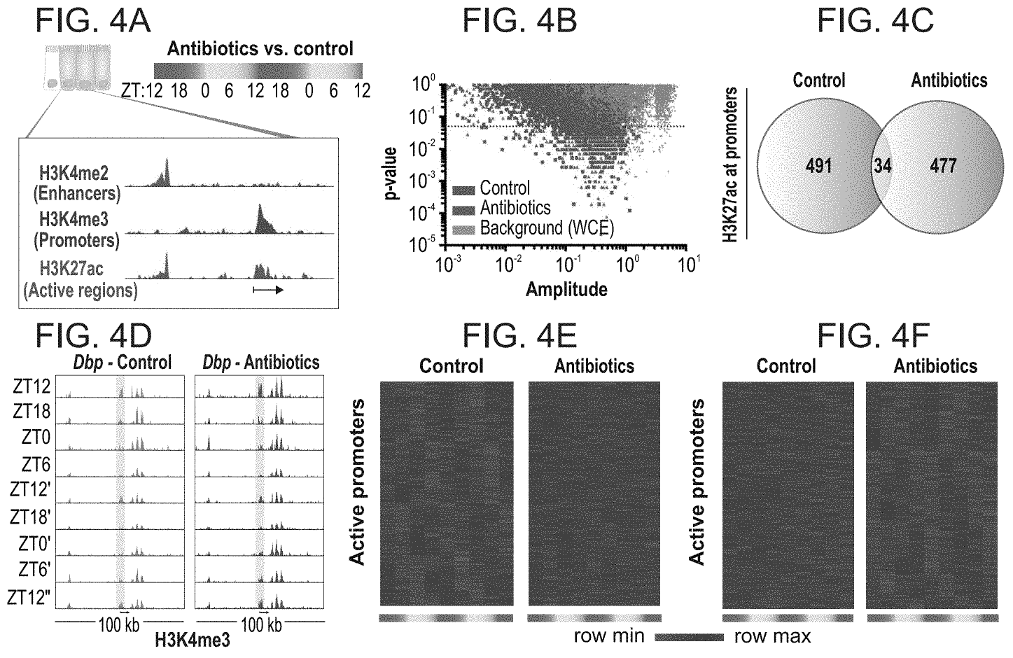

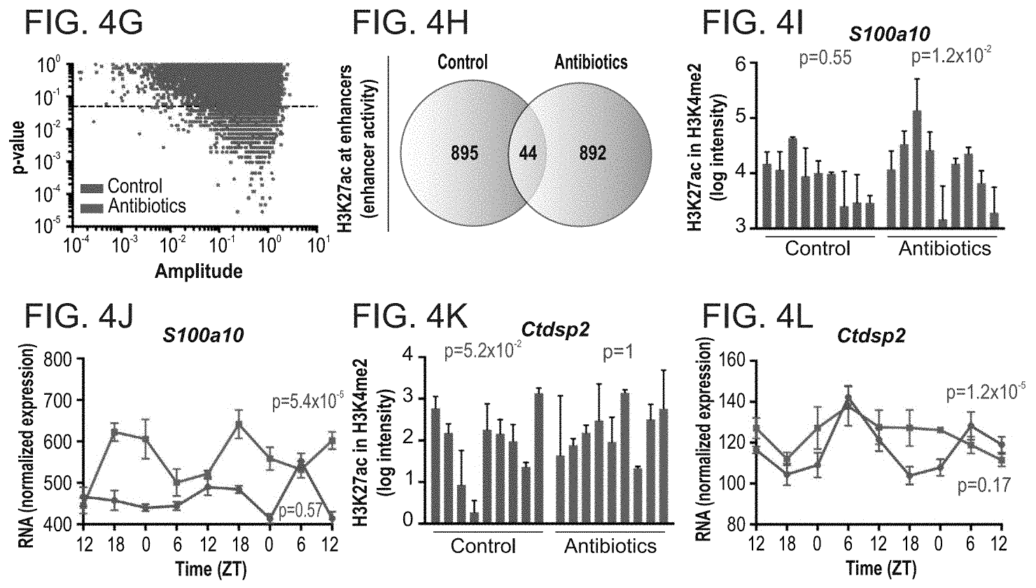

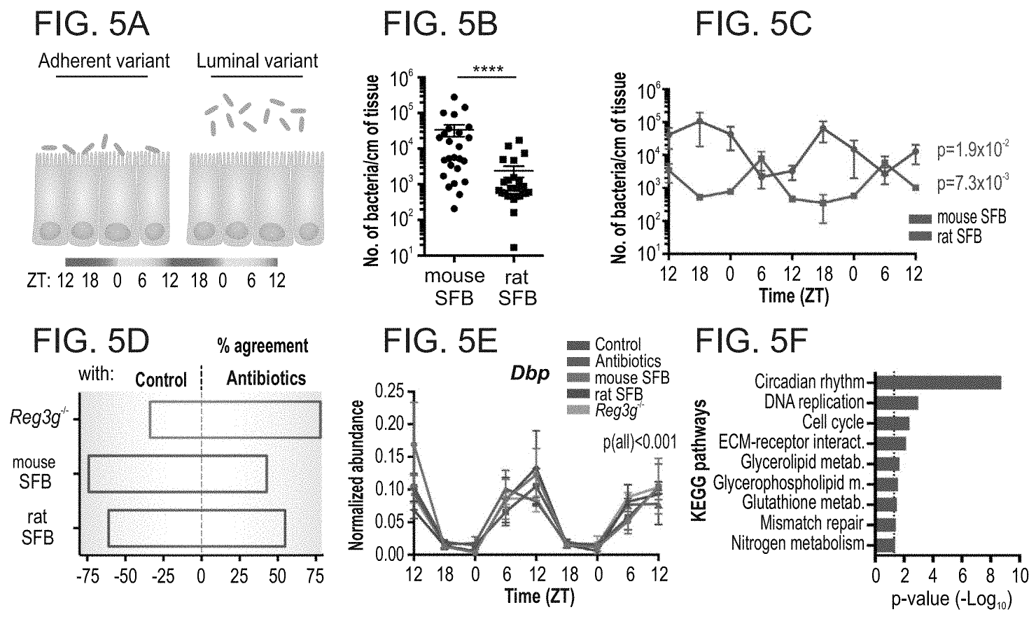

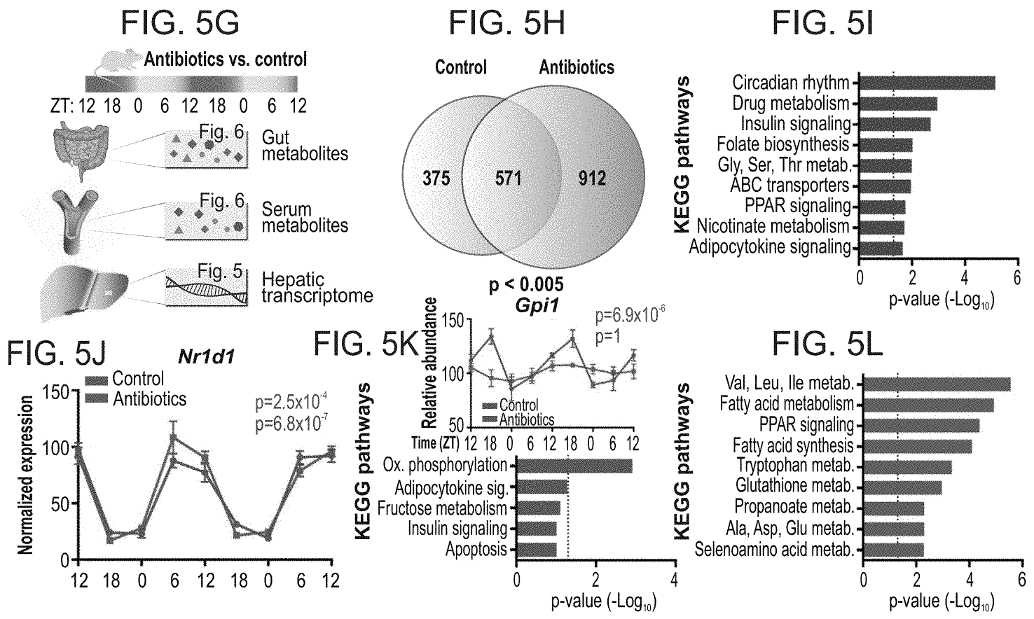

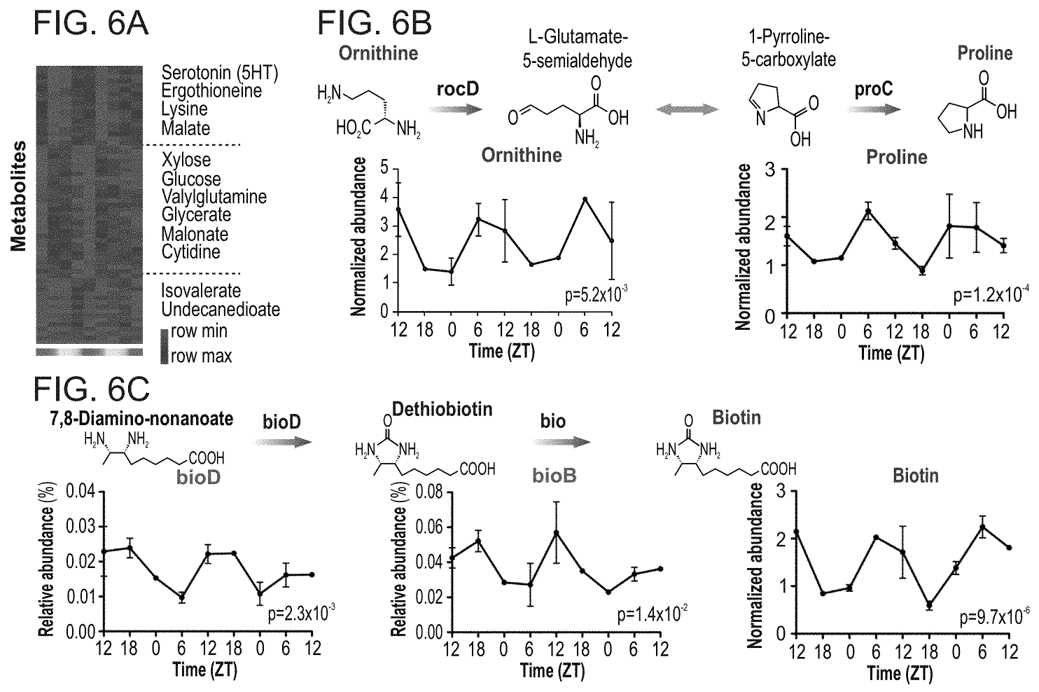

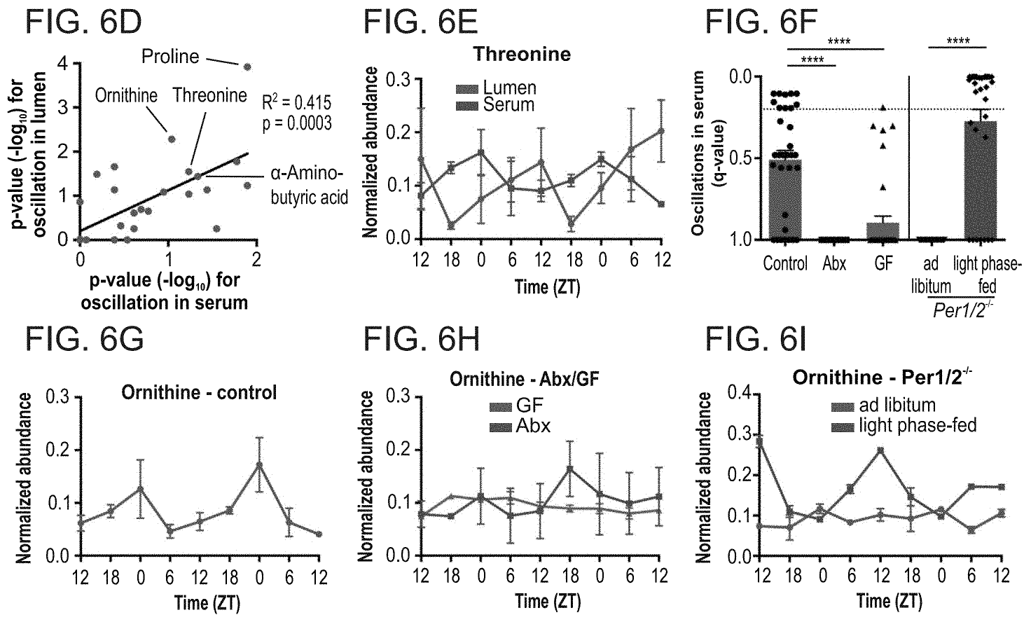

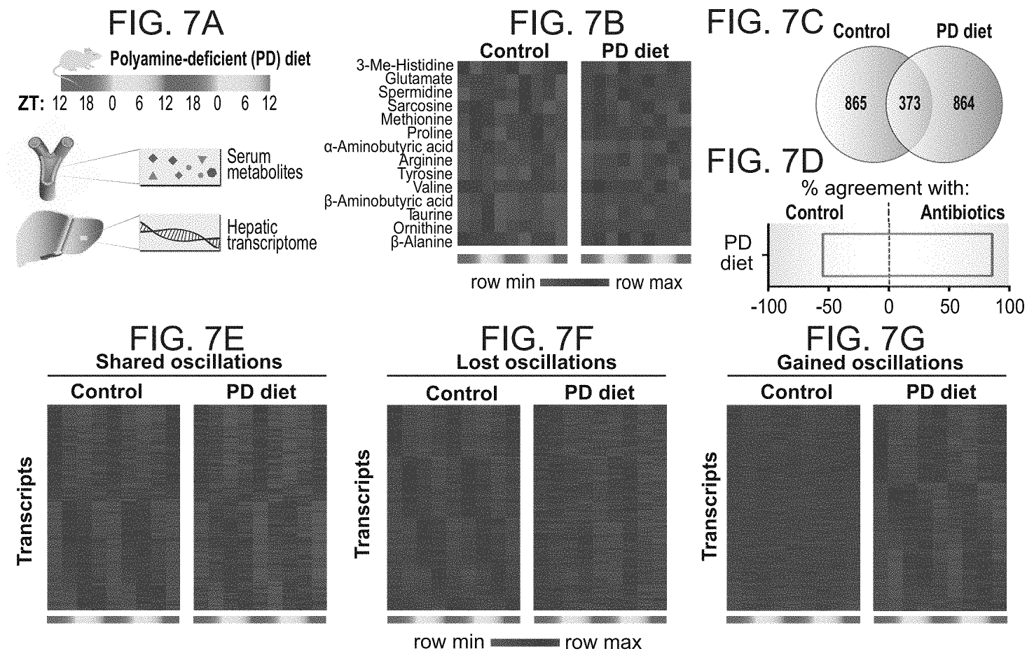

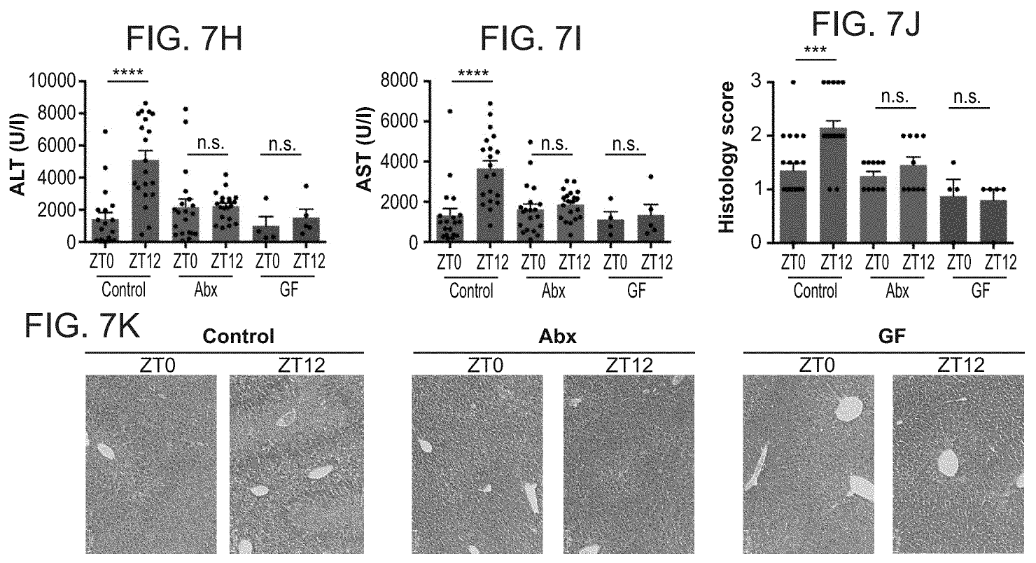

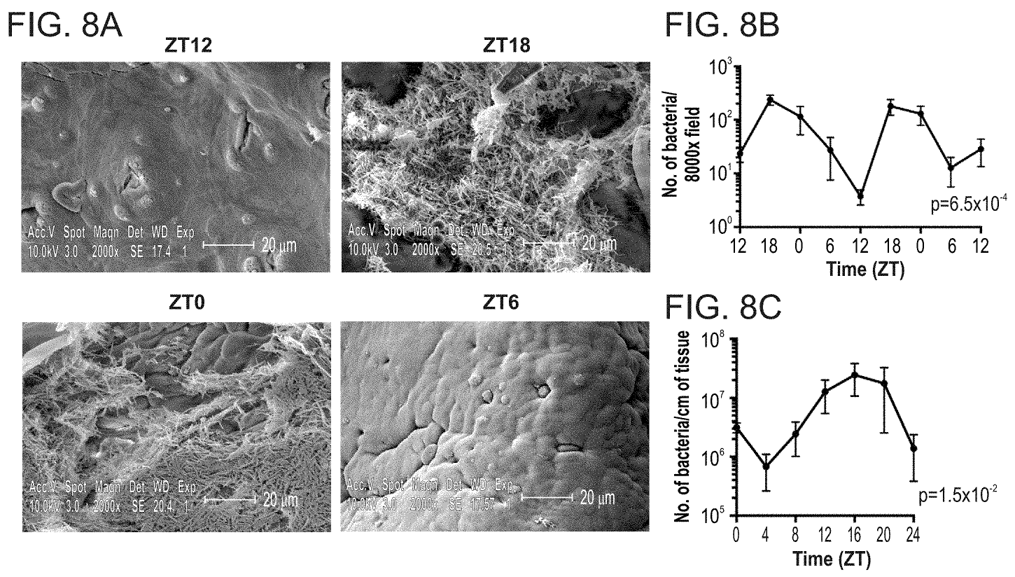

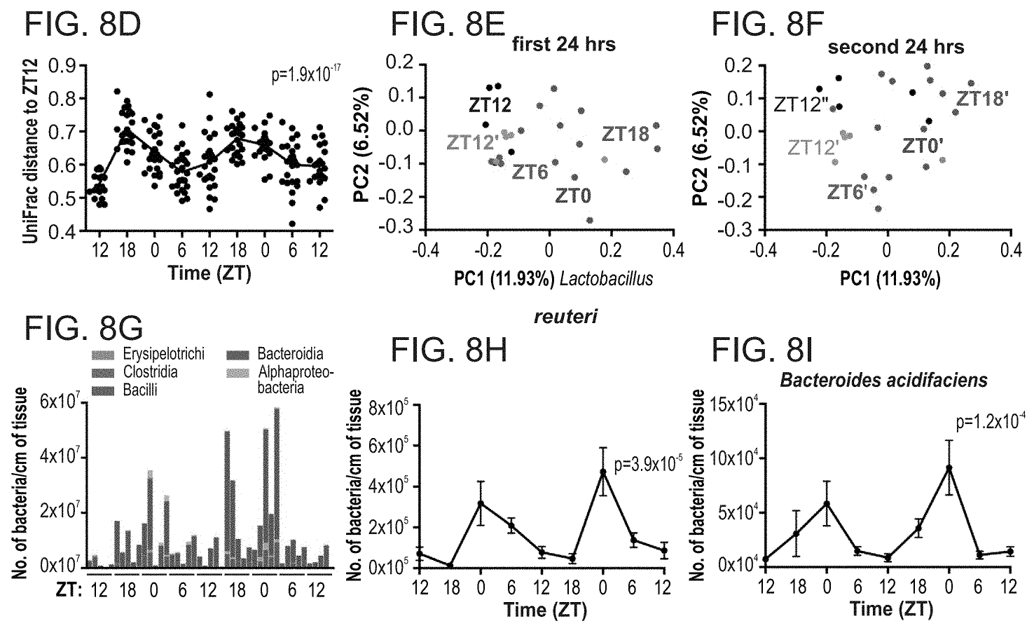

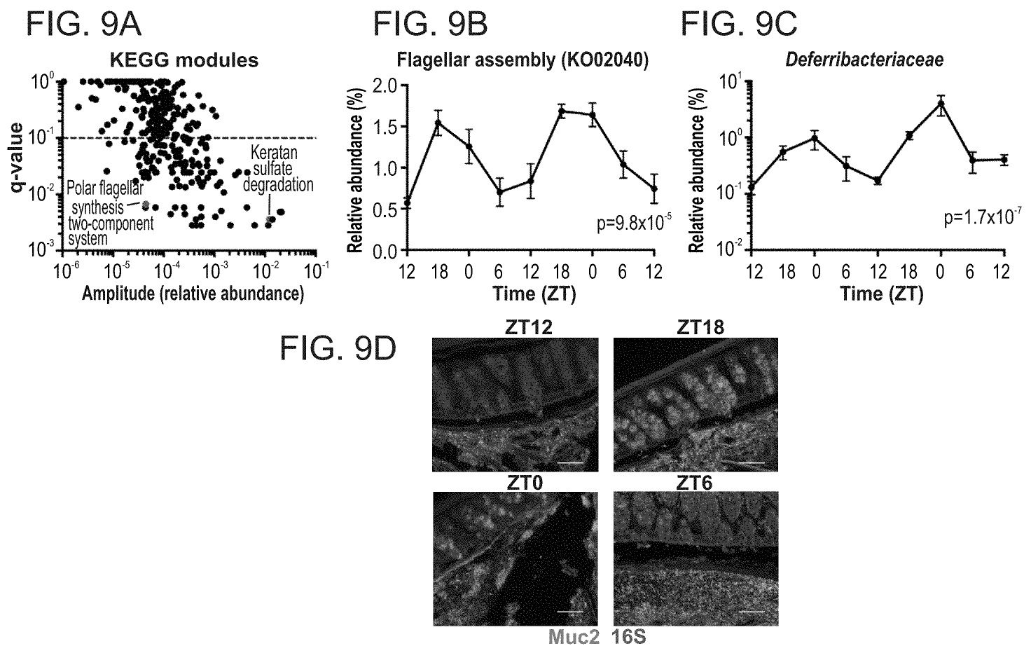

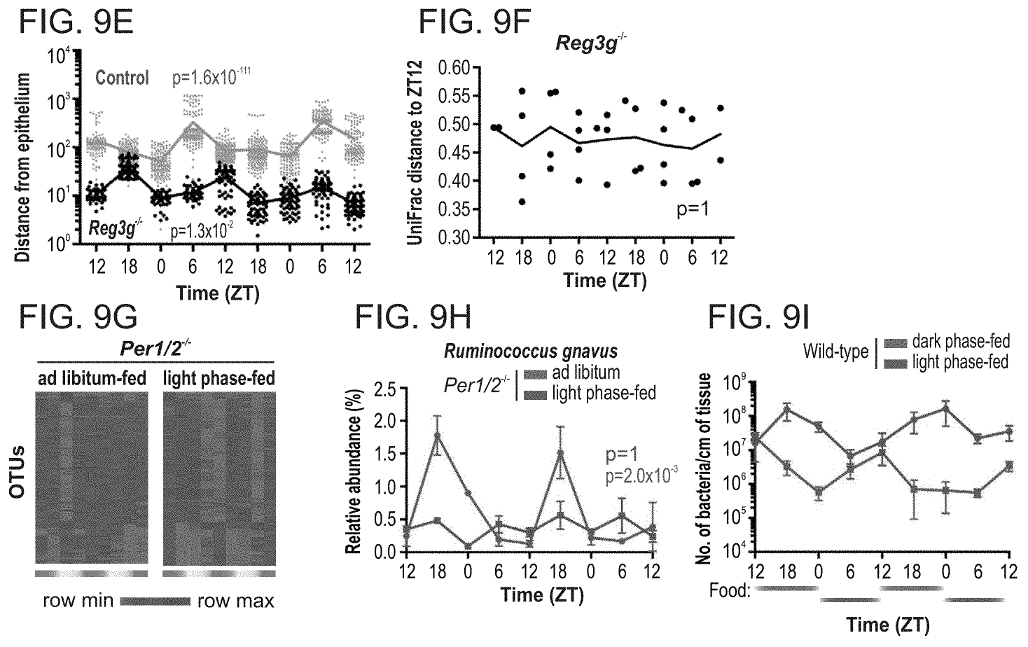

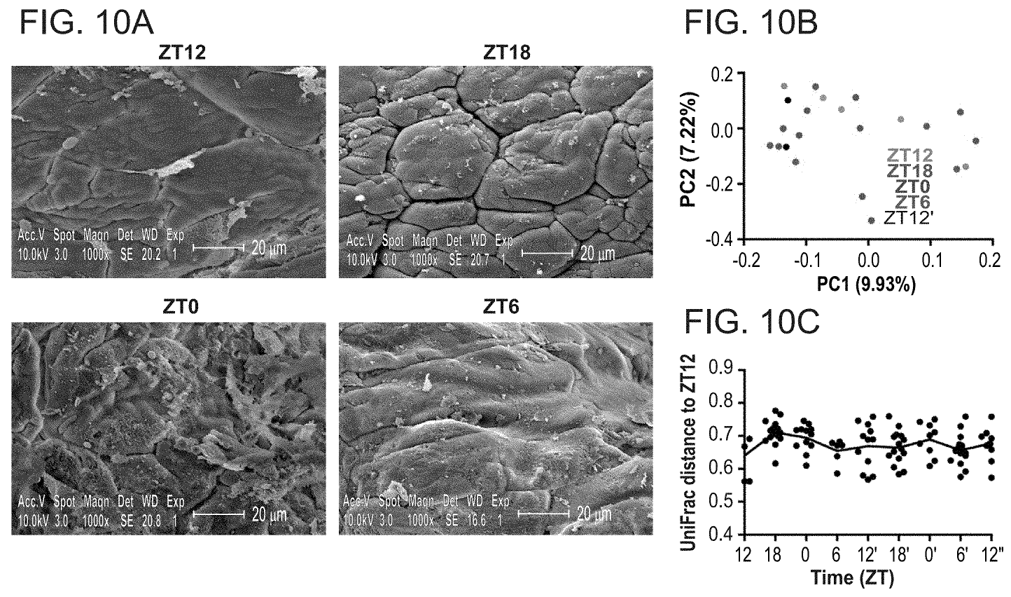

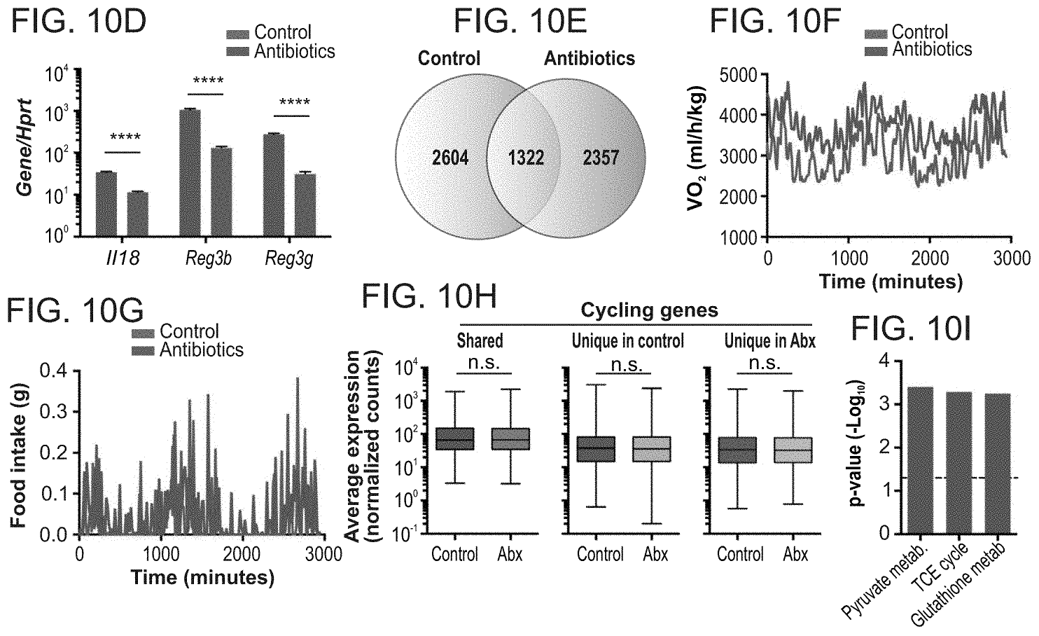

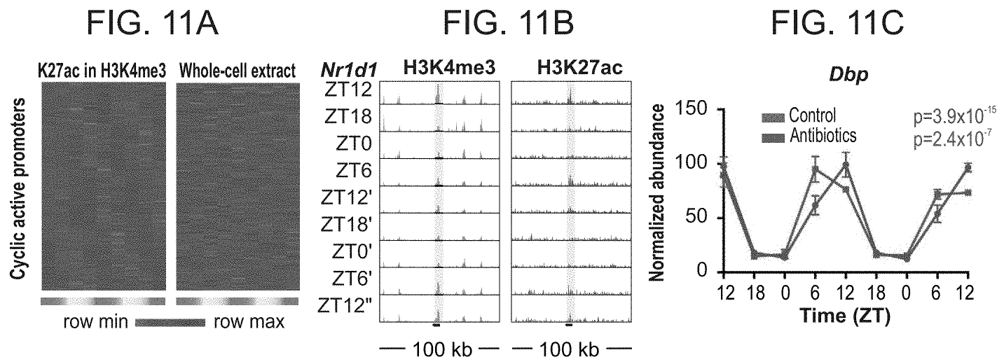

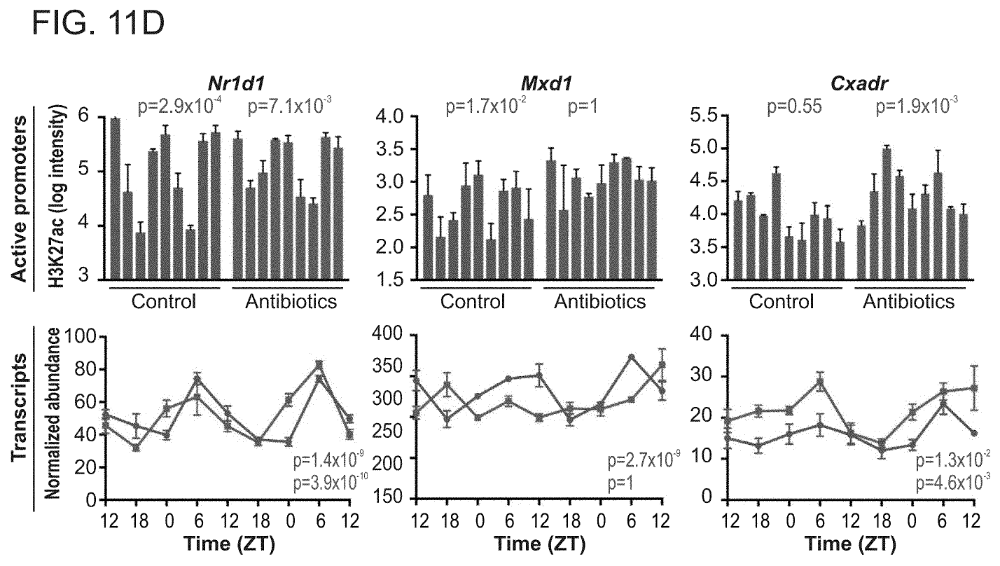

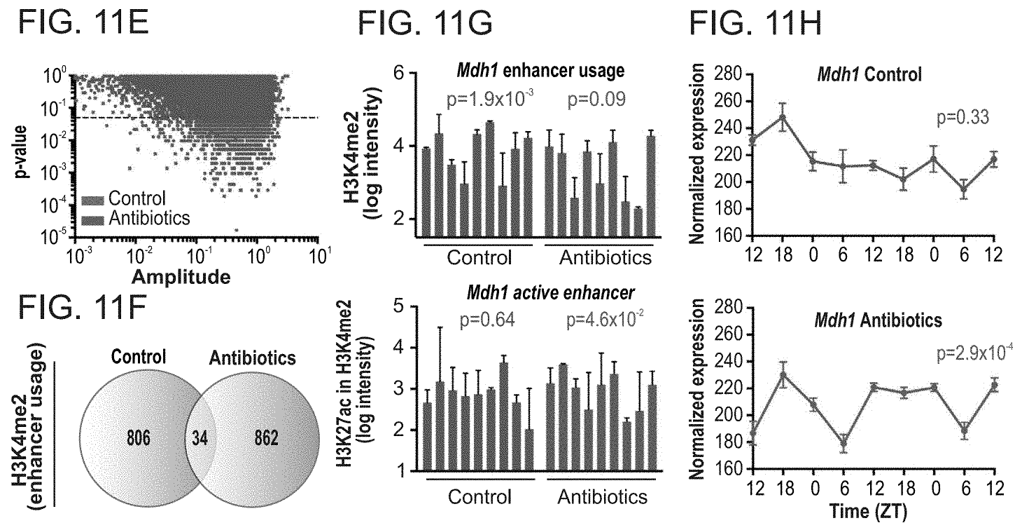

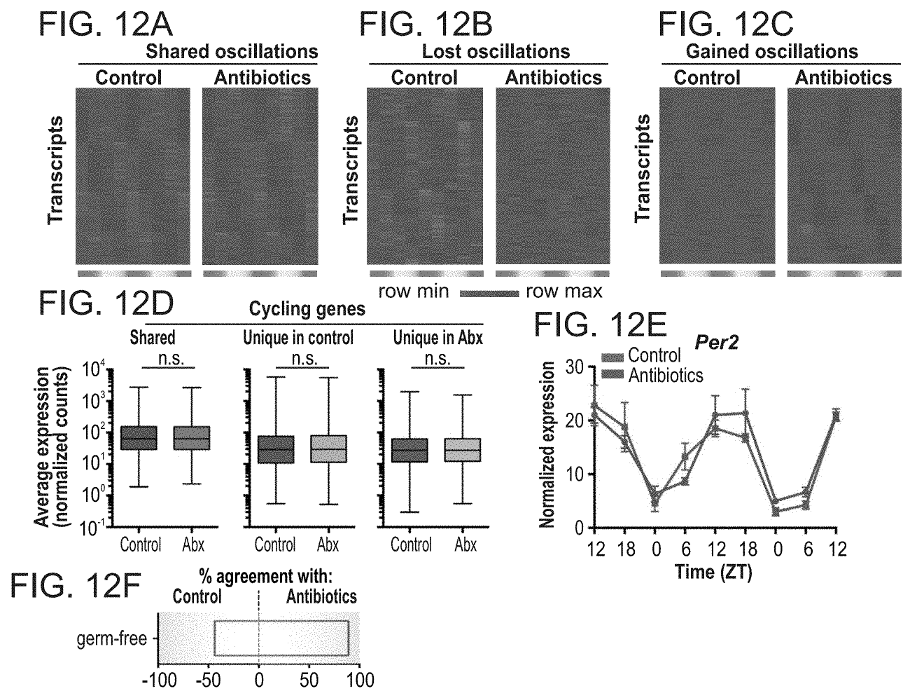

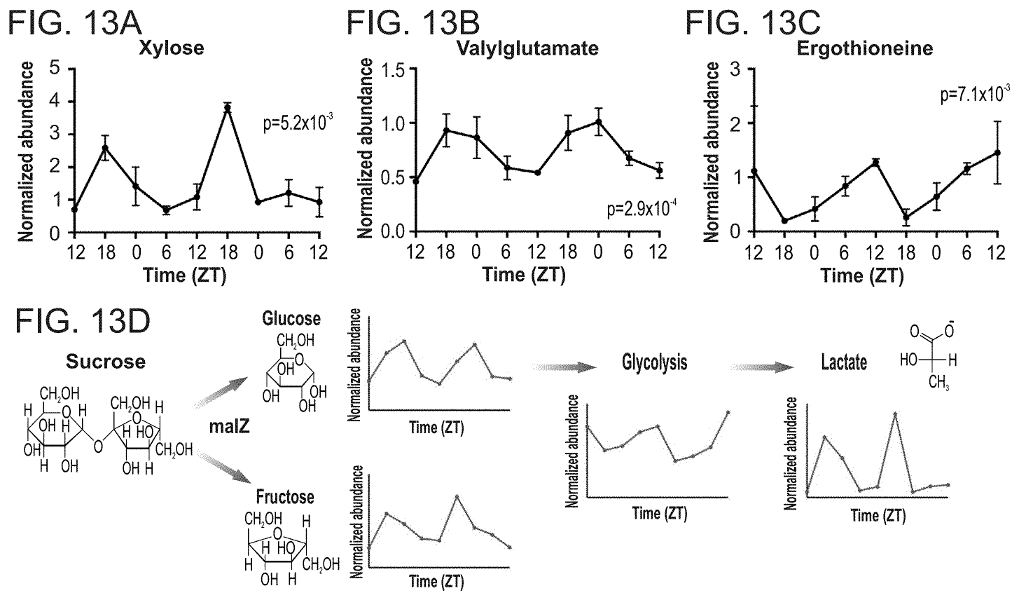

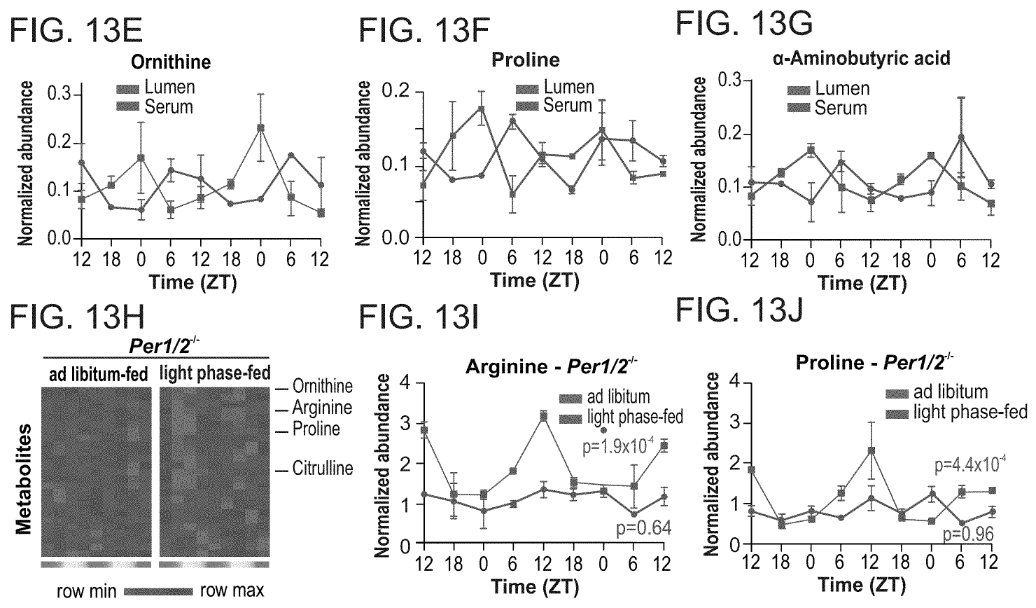

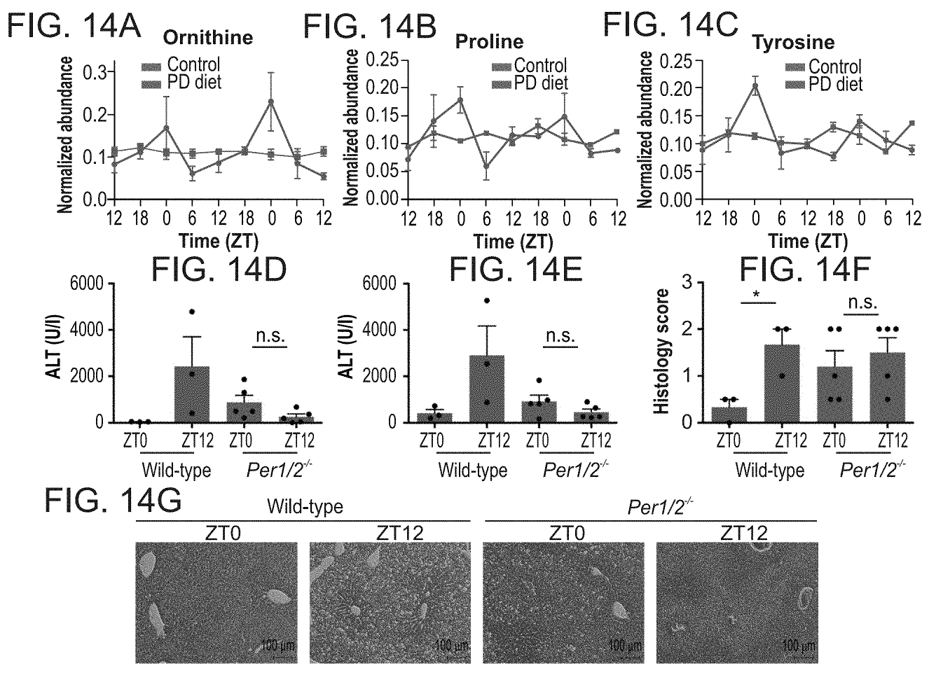

(E) Relative taxonomic composition of mucosal-adherent bacteria in antibiotics-treated mice over the course of two light-dark cycles. See FIG. 1D for comparison to control mice.

(F) Venn diagram of shared and unique oscillating colonic transcripts of antibiotics-treated mice compared to controls, p<0.05 and q<0.1. The sets of oscillating transcripts are significantly non-overlapping (p<0.03).

(G-I) Heatmap representation of shared cycling colonic transcripts between antibiotics-treated mice and controls (G), of transcripts uniquely cycling in control mice (H), and of transcripts uniquely oscillating in antibiotics-treated mice (I), p<0.05 and q<0.1.

(J-L) KEGG analysis of shared cycling colonic transcripts between antibiotics-treated mice and controls (J), of transcripts uniquely cycling in control mice (K), and of transcripts uniquely oscillating in antibiotics-treated mice (L).

Data are representative of two independent experiments with N=27-45 mice. JTK_cycle was used to calculate p- and q-values.

FIGS. 4A-L. Antibiotic treatment reprograms chromatin oscillations in intestinal epithelial cells.

(A) Schematic showing sampling times for epithelial ChIP-seq analysis. Assayed histone marks for enhancers, promoters, and active regions are indicated.

(B) H3K27ac peaks in H3K4me3 regions showing diurnal oscillations in antibiotics-treated and control mice, compared to stochastic oscillations in whole-cell extracts (WCE).

(C) Venn diagram of shared and unique active promoters oscillations in intestinal epithelial cells of antibiotics-treated mice and controls, p<0.05.

(D) Diurnal oscillations in normalized H3K4m3 peaks found in the 100 kb region of the Dbp locus in control (left) and antibiotics-treated mice (right).

(E, F) Heatmaps of active promoter oscillations that are lost (E) and gained (F) upon microbiota depletion.

(G) H3K27ac peaks at enhancers undergoing diurnal oscillations in intestinal epithelial cells of antibiotics-treated and control mice.

(H) Venn diagram of shared and unique active enhancer oscillations in intestinal epithelial cells of antibiotics-treated and control mice.

(I-L) Examples of gained (I, J) or lost (K, L) enhancer activity that correlates with gene expression rhythmicity.

Data are from N=18-45 mice. JTK_cycle was used to calculate p- and q-values. FIGS. 5A-L. Reprogramming of the colonic and hepatic circadian transcriptome.

(A) Schematic luminal and adherent variants of the same bacterial species.

(B) Quantification of mucosal-resident mouse and rat SFB after mono-inoculation of germ-free mice. Data are pooled from several time points. **** denotes p<0.0001 by Mann-Whitney U-test

(C) Numbers of mucosal-resident bacteria over 48 hours in germ-free mice that were mono-colonized with either mouse SFB or rat SFB.

(D) Overlap of cycling genes in the colons of Reg3g mice, mouse SFB-colonized mice, and rat SFB-colonized mice with genes uniquely oscillating in either antibiotics-treated mice or controls.

(E) Rhythmic colonic gene expression of Dbp in the indicated groups.

(F) KEGG analysis of shared cycling colonic transcripts between mouse SFB mono-colonized mice and conventional controls.

(G) Schematic showing sampling times for intestinal and serum metabolite as well as liver transcriptome analysis.

(H) Venn diagram of shared and unique oscillating hepatic transcripts of antibiotics-treated mice compared to controls, p<0.05 and q<0.1. The sets of oscillating transcripts are significantly non-overlapping (p<0.005).

(I) KEGG analysis of shared cycling hepatic transcripts between antibiotics-treated mice and controls.

(J) Rhythmic hepatic gene expression of Nrldl in antibiotics-treated mice and controls.

(K, L) KEGG analysis of hepatic transcripts uniquely cycling in control mice (K) and in antibiotics-treated mice (L). Inset in K shows gene expression of Gpil.

Data are representative of 1-2 independent experiments with N=18-36 mice. JTK_cycle was used to calculate p- and q-values.

FIGS. 6A-I. Diurnal patterns of the microbiota metabolome influence systemic metabolite oscillations.

(A) Heatmap of metabolites undergoing significant oscillations, p<0.05 and q<0.2. Examples for each cluster are indicated.

(B) Depiction of rhythmic elements in the bacterial pathway converting ornithine to proline.

(C) Depiction of rhythmic elements along the bacterial biotin biosynthetic pathway.

(D) Correlation of p-values for rhythmicity of amino acids and polyamines in the intestinal lumen and serum.

(E) Example of metabolites oscillating in intestinal lumen and serum with different phases.

(F) Quantification of significantly oscillating amino acids and polyamines in the sera of controls, antibiotics-treated mice, germ-free mice, ad libitum-fed Per1/2^{-/-} mice, and light phase-fed Per1/2^{-/-} mice.

(G-I) Diurnal oscillations of ornithine abundances in the sera of controls (G), antibiotics-treated and germ-free mice (H), and ad libitum-fed or light phase-fed Per1/2^{-/-} mice (I).

Data are representative 1-2 experiments with N=18 mice. JTK_cycle was used to calculate p- and q-values.

FIGS. 7A-K. The microbiota impacts diurnal liver function and hepatotoxicity.

(A) Schematic showing sampling times for serum metabolites and liver transcriptomes of mice fed a polyamine-deficient (PD) diet.

(B) Heatmap of oscillating serum amino acids and polyamines in mice fed a PD diet and controls.

(C) Venn diagram of shared and unique oscillating hepatic transcripts of mice fed a PD diet compared to controls, p<0.05 and q<0.1.

(D) Overlap of cycling genes in the livers of mice on a PD diet with genes uniquely oscillating in either antibiotics-treated mice or controls.

(E-G) Heatmap representation of shared cycling colonic transcripts between mice fed a PD diet and controls (G), of transcripts uniquely cycling in control mice (H), and of transcripts uniquely oscillating in PD diet mice (I), p<0.05 and q<0.1.

(H-K) Serum levels of liver enzymes (H, I), liver histology score (J), and representative histological images (K) of control, antibiotics-treated (Abx), and germ-free mice (GF) that were injected with APAP at ZTO or ZT12. ** denotes p<0.01 and **** denotes p<0.0001 by Mann-Whitney U-test. Scale bars indicate 100 μm.

Data in A-G were obtained with N=18-36 mice. Data in H-K are representative of 3 independent experiments with N=4-10 mice in each groups. JTK_cycle was used to calculate p- and q-values.

FIGS. 8A-I. Diurnal fluctuations in the number and composition of mucosal-associated commensals.

(A, B) SEM images (A) and quantification (B) showing diurnal fluctuations in epithelial colonization by bacteria. Images are representative of 10 randomly chosen views per mouse.

(C) Diurnal fluctuations in the number of bacteria attached to colonic epithelium determined every 4 hours over one light-dark cycle.

(D) Unweighted UniFrac distances of the initial time point compared to all other time points over the course of two light-dark cycles.

(E, F) Diurnal rhythmicity of beta-diversity of mucosal-adherent bacteria, as shown by PCoA of samples obtained from two consecutive dark-light phases. ZT12′ indicates ZT12 on the following day.

(G) Absolute numbers of taxonomic composition of mucosal-adherent bacteria over the course of two light-dark cycles.

(H, I) Examples of bacterial species showing fluctuating absolute numbers in mucosal-adherent communities.

Data are representative of two independent experiments with N=45 mice. Panel C is from one experiment with N=35 mice. JTK_cycle was used to calculate p-values.

FIGS. 9A-I. Diurnal fluctuations in bacterial motility and mucus degradation.

(A) KEGG modules of the mucosal microbial community showing diurnal oscillations in relative abundance. Selected modules and genes are highlighted in red.

(B) Diurnal fluctuations in the relative abundance of flagellar assembly in mucosal-associated communities.

(C) Diurnal fluctuations in the relative abundance of Deferribacteriaceae in mucosal-associated communities.

(D) Immunofluorescence images showing diurnal fluctuations in epithelial proximity by commensal bacteria. Stained are Muc2 protein (green) and bacterial 16S rDNA (red). Images are representative of 10 randomly chosen views per mouse. Scale bars indicate 100 μm.

(E) Quantification of diurnal fluctuations in epithelial proximity by commensal bacteria in Reg3g mice. Wild-type controls are shown in grey for comparison.

(F) Unweighted UniFrac distances of the initial time point compared to all other time points in Reg3g^{-/-} mice over the course of two light-dark cycles. See Figure FIG. 8D for comparison to wild-type mice.

(G, H) Heatmap (G) and representative example (H) of oscillating OTUs in Per1/2^{-/-} mice that were either fed ad libitum or only during the light phase.

(I) Diurnal fluctuations in the number of bacteria attached to colonic epithelium in wild-type mice fed either during the dark phase or during the light phase.

Data are representative of 1-2 independent experiments with N=18-45 mice. JTK_cycle was used to calculate p- and q-values.

FIGS. 10A-I. The microbiota is required for coordinated oscillations in the intestinal transcriptome.

(A) Representative SEM images of epithelial colonization by bacteria over the course of a day in antibiotics-treated mice. See FIG. 1B for comparison to control mice.

(B) PCoA of mucosal-adherent communities in antibiotics-treated mice every 6 hours over the course of one day. See FIGS. 8E and 8F for comparison to control mice.

(C) UniFrac distance of the initial time point compared to all other time points over the course of two light-dark cycles in antibiotics-treated mice. See FIG. 8D for comparison to control mice.

(D) Colonic expression of 1118, Reg3b, and Reg3g in antibiotics-treated mice and controls. Data are pooled from several time points. **** denotes p<0.0001 by Mann-Whitney U-test

(E) Venn diagram of shared and unique oscillating colonic transcripts of antibiotics-treated mice compared to controls sampled every 4 hours over 24 hours.

(F, G) 48-hour recordings of rhythmic oxygen consumption (F) and food intake (G) in antibiotics-treated mice and controls.

(H) Average colonic expression levels in antibiotics-treated mice and controls of genes that are cycling in either both conditions (shared), only in controls, or only in antibiotics (Abx)-treated mice.

(I) Rhythmicity analysis in the mucosal microbiota of KEGG pathways that acquire rhythmicity in colonic transcripts upon antibiotic treatment.

Data are representative of 1-2 independent experiments with N=45 mice.

FIGS. 11A-H. The impact of the microbiota on the diurnal epigenetic landscape.

(A) Heatmap representation of cycling active promoter marks compared to genomic background in whole-cell extracts.

(B) Diurnal oscillations in normalized H3K4me3 peaks (left) and H3K27ac peaks (right) found in the 100 kb region of the Nrldl locus.

(C) Rhythmic colonic gene expression of Dbp in control and antibiotics-treated mice.

(D) Examples of shared (left), lost (middle), and gained (right) oscillations in active promoters (above) and gene expression (below) in antibiotics-treated mice compared to controls.

(E) H3K4me2 peaks at enhancers undergoing diurnal oscillations in intestinal epithelial cells of antibiotics-treated and control mice. Dashed line indicates p<0.05.

(F) Venn diagram of shared and unique enhancer usage oscillations in intestinal epithelial cells of antibiotics-treated and control mice.

(G, H) Example of gene with H3K4me2 peak oscillation in the control group (G), but H3K27ac oscillation at enhancer (G) and rhythmic gene expression (H) uniquely in antibiotics-treated mice.

Data are from N=18-45 mice. JTK_cycle was used to calculate p- and q-values.

FIGS. 12A-F. The impact of the microbiota on hepatic transcriptome oscillations.

(A-C) Heatmap representation of shared cycling hepatic transcripts between antibiotics-treated mice and controls (A), of transcripts uniquely cycling in control mice (B), and of transcripts uniquely oscillating in antibiotics-treated mice (C), p<0.05 and q<0.1.

(D) Hepatic expression levels in antibiotics-treated mice and controls of genes that are cycling in either both conditions (shared), only in controls, or only in antibiotics-treated mice.

(E) Rhythmic hepatic gene expression of Per2 in antibiotics-treated mice and controls.

(F) Overlap of cycling genes in the livers of germ-free mice with genes uniquely oscillating in either antibiotics-treated mice or controls.

Data are representative of 1-2 independent experiments with N=18-36 mice. JTK_cycle was used to calculate p- and q-values.

FIGS. 13A-J. Intestinal and systemic metabolite oscillations.

(A-C) Examples of metabolites oscillating in the intestinal lumen.

(D) Depiction of rhythmic elements along the bacterial sucrose degradation pathway.

(E-G) Examples of amino acids oscillating in intestinal lumen and serum.

(H-J) Heatmap (H) and examples (I, J) of amino acids and polyamines in the serum of ad libitum-fed or light phase-fed Per1/2^{-/-} mice.

Data are from 1-2 experiments with N=18 mice. JTK_cycle was used to calculate p- and q-values.

FIGS. 14A-G. The impact of the microbiota and metabolites on circadian liver function.

(A-C) Examples of serum amino acids and polyamines losing oscillations upon feeding with a polyamine-deficient (PD) diet.

(D-G) Serum levels of liver enzymes (D, E) liver histology score (F), and representative histological images (G) from Per1/2^{-/-} mice and wild-type controls that were injected with APAP at ZTO or ZT12.

Data are representative of 1-2 independent experiments with N=18-36 mice.

### DESCRIPTION OF SPECIFIC EMBODIMENTS OF THE INVENTION

The present invention, in some embodiments thereof, relates to methods of treating liver toxicity and disorders. The methods rely on altering metabolite rhythms and programming of transcriptional oscillations of the liver via manipulation of the circadian activity of the host's gut microbiome.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The intestinal microbiota undergoes diurnal compositional and functional oscillations that affect metabolic homeostasis (see for example WO2015/166492), but the mechanisms by which the rhythmic microbiota influences host circadian activity remain elusive. Using integrated multi-omics and imaging approaches, the present inventors now demonstrate that the gut microbiota features oscillating biogeographical localization and metabolome patterns that determine the rhythmic exposure of the intestinal epithelium to different bacterial species and their metabolites over the course of a day.

This diurnal microbial behavior drives, in turn, the global programming of the host circadian transcriptional, epigenetic, and metabolite oscillations. Surprisingly, disruption of homeostatic microbiome rhythmicity not only abrogates normal chromatin and transcriptional oscillations of the host, but also incites genome-wide de novo oscillations in both intestine and liver, thereby impacting diurnal fluctuations of host physiology and disease susceptibility. As such, the rhythmic biogeography and metabolome of the intestinal microbiota regulates the temporal organization and functional outcome of host transcriptional and epigenetic programs.

In light of these findings, the present inventors propose that the circadian fluctuations in intestinal microbiota may impact the homeostatic diurnal variation in hepatic drug detoxification and hepatotoxicity.

Whilst reducing the present invention to practice the present inventors analyzed the effect of disruption of microbiota diurnal rhythmicity on circadian liver function.

The time of day is known to greatly affect hepatic drug metabolism, including the detoxification of acetaminophen (acetyl-para-aminophenol, APAP). The present inventors therefore administered APAP at different circadian times (ZTO versus ZT12) and assessed APAP-induced hepatotoxicity by the measurement of liver enzyme release as well as liver histology. As illustrated in FIGS. 7H-K, mice featured dramatically exacerbated liver toxicity when APAP was injected at ZT12 as compared to ZTO (FIGS. 7H-7K). Remarkably, mice with a disrupted microbiota diurnal rhythmicity (e.g. antibiotics-treated or germ-free mice) lost this diurnal variation in the severity of APAP-induced hepatotoxicity, and featured low and comparable levels of liver damage at different times of the day (FIG. 7H-7K), manifesting as a significantly lower aminotransferase activity, reduced liver necrosis, and improved histopathological score. Together, these results suggest that homeostatic microbiota rhythms and microbiota-mediated maintenance of the circadian transcriptome is necessary to maintain normal diurnal activity in hepatic drug metabolism.

Thus, according to a first aspect of the present invention there is provided a method of treating a liver disease in a subject in need thereof comprising administering to the subject:

(i) a therapeutically effective amount of an agent which is therapeutic for the liver disease; and

(ii) an agent which alters the circadian rhythmicity of microbes of the gut microbiome of the subject, thereby treating the liver disease.

The term “treating” refers to inhibiting, preventing or arresting the development of a live disease, disorder or condition, and/or causing the reduction, remission, or regression of the disease. Those of skill in the art will understand that various methodologies and assays can be used to assess the development of a pathology, and similarly, various methodologies and assays may be used to assess the reduction, remission or regression of the pathology.

As used herein, the term “preventing” refers to preventing the liver disease from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology.

As used herein, the term “liver disease” refers to any disease or disorder that affects the liver. The liver disease may be acute or chronic. Typically, the liver disease causes damage to the liver. The amount of damage to the liver is typically based on the duration of the disease. The liver damage may be caused by infection, injury, exposure to drugs or toxic compounds such as alcohol or impurities in foods, an abnormal build-up of normal substances in the blood, an autoimmune process, a genetic defect (such as haemochromatosis), or other unknown causes.

In one embodiment, the liver disease is a chronic liver disease which involves a cirrhotic liver and/or a fibrotic liver. In one preferred embodiment, the chronic liver disease is cirrhosis.

Chronic liver disease or cirrhosis may be caused by or be associated with various medical conditions. Without being limited thereto, such conditions may include chronic alcohol consumption, chronic liver infection, chronic liver inflammation, inflammatory disorder, autoimmune disorder, drug-induced liver injury, nonalcoholic metabolic disorder, chronic liver injury, hepatocellular carcinoma, adverse hepatic reactions upon use of dietary supplements.

Liver infection may be of any type known in the art to cause injury to liver tissue. In some embodiments, the chronic liver is chronic hepatitis B, C or D. Chronic hepatitis B or C cause inflammation that over time damages the liver and leads to cirrhosis. Hepatitis D also causes cirrhosis, but will occur only in subjects who already have hepatitis B.

According to some embodiments, the chronic liver infection is HIV infection, cytomegalovirus or Epstein-Barr virus.

The liver disease may also be drug induced as a result of long term drug treatment. Drugs inducing liver damage may be grouped according to the type of enzyme manifesting marked level elevation (indicative of liver damage). For example, hepatocellular hepatotoxicity, which may be a result from drugs such as acetaminophen and isoniazid; Cholestatic hepatotoxicity which is characterized by development of pruritus and jaundice accompanied by marked elevation of serum alkaline phosphatase levels, may be a result from drugs such amoxicillin/clavulanic acid and chlorpromazine.

Particular examples of drugs that may cause liver disease include Amiodarone, Methotrexate and Nitrofurantoin.

According to a particular embodiment, the liver disease is an inflammatory or autoimmune disorder. The inflammatory or autoimmune disorder may be selected, without being limited thereto, chronic pancreatitis, inflammatory bowel disease, primary sclerosing cholangitis, primary biliary cirrhosis, primary hepatothiasis and recurrent pyogenic cholangitis, systemic lupus erythematosus, Celiac disease, hypothyroidism, Raynaud's phenomenon, Sicca syndrome.

According to other embodiments, the liver disease is a metabolic disorder, such as, without being limited thereto, diabetes, obesity, steatosis and non-alcoholic steatohepatitis, haemochromatosis, cystic fibrosis, alpha-1 antitrypsin deficiency, galactosemia, glycogen storage diseases, Wilson's disease, and intrahepatic cholestasis. Some of these causes are also recognized as inherited diseases.

Other exemplary liver diseases include, but are not limited to, cirrhosis, liver fibrosis, non-alcoholic fatty liver disease (NAFLD), non-alcoholic steatohepatitis (NASH), hepatic ischemia reperfusion injury, primary biliary cirrhosis (PBC), primary sclerosing cholangitis (PSC and hepatitis, including both viral and alcoholic hepatitis.

Non-alcoholic fatty liver disease (NAFLD) is the build up of extra fat in liver cells that is not caused by alcohol. NAFLD may cause the liver to swell (i.e. steatohepatitis), which in turn may cause scarring (i.e. cirrhosis) over time and may lead to liver cancer or liver failure. NAFLD is characterized by the accumulation of fat in hepatocyes and is often associated with some aspects of metabolic syndrome (e.g. type 2 diabetes mellitus, insulin resistance, hyperlipidemia, hypertension). The frequency of this disease has become increasingly common due to consumption of carbohydrate-rich and high fat diets. A subset (.about.20%) of NAFLD patients develop nonalcoholic steatohepatitis (NASH).

NASH, a subtype of fatty liver disease, is the more severe form of NAFLD. It is characterized by macrovesicular steatosis, balloon degeneration of hepatocytes, and/or inflammation ultimately leading to hepatic scarring (i.e. fibrosis). Patients diagnosed with NASH may progress to advanced stage liver fibrosis and eventually cirrhosis. Once NASH is developed, it could cause the liver to undergo destructive remodeling leading to scarring (i.e. cirrhosis) over time. The current treatment for cirrhotic NASH patients with end-stage disease is liver transplant.

Other examples of liver disease include, but are not limited to bile acid synthesis disorders, Fascioliasis; Hepatitis; Alcoholic liver disease; Fatty liver disease; Cirrhosis; liver; biliary; sclerosing cholangitis; Centrilobular necrosis; Budd-Chiari syndrome; Hereditary liver diseases (hemochromatosis, involving accumulation of iron in the body, and Wilson's disease); transthyretin-related hereditary amyloidosis; and Gilbert's syndrome. Additional examples include, but are not limited to Alagille Syndrome; Alcohol-Related Liver Disease; Alpha-1 Antitrypsin Deficiency; Autoimmune Hepatitis; Benign Liver Tumors; Biliary Atresia; Cirrhosis; Galactosemia; Gilbert Syndrome; Hemochromatosis; Hepatitis A; Hepatitis B; Hepatitis C; Hepatocellular Carcinoma; Hepatic Encephalopathy; hepatic coma, Liver Cysts; Liver Cancer; Newborn Jaundice; Non-Alcoholic Fatty Liver Disease (including nonalcoholic fatty liver and nonalcoholic steatohepatitis); Primary Biliary Cirrhosis (PBC); Primary Sclerosing Cholangitis (PSC); Reye Syndrome; Type I Glycogen Storage Disease and Wilson Disease.

Examples of agent useful for treating liver diseases include but are not limited to anti-viral drugs, colchicine, corticosteroids, curcumin, glycyrrhizin, interferons (for their antifibrotic properties), Liv 52, nitric oxide, resveratrol, silymarin, sulfoadenosylmethionine, and thalidomide, As used herein, the term “microbiome” refers to the totality of microbes (bacteria, fungae, protists), their genetic elements (genomes) in a defined environment.

According to a particular embodiment, the agent for treating the liver disease is not an antibiotic.

More specifically, examples of agents that are used to treat bile acid synthesis disorders include, but are not limited to cholbam and cholic acid.

Examples of agents for treating Hyperbilirubinemia include phenobarbital and luminal.

Examples of agents for treating Liver Cirrhosis include, but are not limited to ursodiol, Actigall, Urso, Urso Forte, colchinine, azathioprine, obeticholic acid and Ocaliva.

Examples of agents for treating liver metastasis in adenocarcinoma include FUDR and floxuridine.

Examples of agents for treating non-alcoholic fatty liver disease include, but are not limited to ursodiol, Actos, Actigall, Avandia, Pioglitazaone, Cystadane, Urso, Urso Forte, olistat, betaine and rosiglitazone.

Examples of agents for treating portal hypertension include but are not limited to propranolol and nadolol.

As mentioned, as well as providing agents which are therapeutic for treating the liver disease, the present inventors further contemplate administering to the subject agents which alters the circadian rhythmicity of microbes of the gut microbiome of the subject in order to treat the liver disease.

As used herein, the term “microbiome” refers to the totality of microbes (bacteria, fungae, protists), their genetic elements (genomes) in a defined environment (e.g. gut).

In one embodiment, the defined environment is the intestine (small and/or large intestine)—i.e. gut. In one embodiment, the defined environment is the intestinal mucosa. In another embodiment, the defined environment is the feces.

According to a particular embodiment, the agent alters the circadian rhythmicity of at least one microbe (e.g. bacteria) of the gut microbiome.

According to a particular embodiment, the agent alters the circadian rhythmicity of at least 5 microbes (e.g. bacteria) of the gut microbiome.

According to a particular embodiment, the agent alters the circadian rhythmicity of at least 10 microbes (e.g. bacteria) of the gut microbiome.

According to a particular embodiment, the agent alters the circadian rhythmicity of at least 20 microbes (e.g. bacteria) of the gut microbiome.

According to a particular embodiment, the agent alters the circadian rhythmicity of at least 50 microbes (e.g. bacteria) of the gut microbiome.

According to a particular embodiment, the agent alters the circadian rhythmicity of at least 100 microbes (e.g. bacteria) of the gut microbiome.

Examples of particular bacteria whose abundance is known to fluctuate with a circadian rhythmicity include for example Mucispirillum schaedleri, Lactobacillus reuteri, Bacteroides acidifaciens, Deferribacteraceae, Wolinella succinogenes, Nitratiruptor, Helicobacter bizzozeronii CIII-1, Denitrovibrio acetiphilus DSM 12809, Calditerrivibrio nitroreducens DSM 19672, Deferribacter desulfuricans SSM], Desulfurispirillum indicum S5, Selenomonas ruminantium subsp. lactilytica TAM6421, Hippea maritima DSM 10411, Nautilia profundicola AmH, Wolinella succinogenes, Campylobacter lari RM2100, Sulfurospirillum deleyianum DSM 6946, Candidatus Sulfuricurvum sp. RIFRC-1, Pelobacter carbinolicus DSM 2380, Desulfohalobium retbaense DSM 5692 and The rmodesulfatator indicus DSM 15286.

The present inventors have shown that antibiotic agents are capable of altering the circadian rhythmicity of bacteria.

As used herein, the term “antibiotic agent” refers to a group of chemical substances, isolated from natural sources or derived from antibiotic agents isolated from natural sources, having a capacity to inhibit growth of, or to destroy bacteria, and other microorganisms, used chiefly in treatment of infectious diseases. Examples of antibiotic agents include, but are not limited to; Amikacin; Amoxicillin; Ampicillin; Azithromycin; Azlocillin; Aztreonam; Aztreonam; Carbenicillin; Cefaclor; Cefepime; Cefetamet; Cefinetazole; Cefixime; Cefonicid; Cefoperazone; Cefotaxime; Cefotetan; Cefoxitin; Cefpodoxime; Cefprozil; Cefsulodin; Ceftazidime; Ceftizoxime; Ceftriaxone; Cefuroxime; Cephalexin; Cephalothin; Cethromycin; Chloramphenicol; Cinoxacin; Ciprofloxacin; Clarithromycin; Clindamycin; Cloxacillin; Co-amoxiclavuanate; Dalbavancin; Daptomycin; Dicloxacillin; Doxycycline; Enoxacin; Erythromycin estolate; Erythromycin ethyl succinate; Erythromycin glucoheptonate; Erythromycin lactobionate; Erythromycin stearate; Erythromycin; Fidaxomicin; Fleroxacin; Gentamicin; Imipenem; Kanamycin; Lomefloxacin; Loracarbef; Methicillin; Metronidazole; Mezlocillin; Minocycline; Mupirocin; Nafcillin; Nalidixic acid; Netilmicin; Nitrofurantoin; Norfloxacin; Ofloxacin; Oxacillin; Penicillin G; Piperacillin; Retapamulin; Rifaxamin, Rifampin; Roxithromycin; Streptomycin; Sulfamethoxazole; Teicoplanin; Tetracycline; Ticarcillin; Tigecycline; Tobramycin; Trimethoprim; Vancomycin; combinations of Piperacillin and Tazobactam; and their various salts, acids, bases, and other derivatives. Anti-bacterial antibiotic agents include, but are not limited to, aminoglycosides, carbacephems, carbapenems, cephalosporins, cephamycins, fluoroquinolones, glycopeptides, lincosamides, macrolides, monobactams, penicillins, quinolones, sulfonamides, and tetracyclines.

According to a particular embodiment, the antibiotic is Ampicillin, Neomycin, Vancomycin or Mettronidazole.

Antibacterial agents also include antibacterial peptides. Examples include but are not limited to abaecin; andropin; apidaecins; bombinin; brevinins; buforin II; CAP18; cecropins; ceratotoxin; defensins; dermaseptin; dermcidin; drosomycin; esculentins; indolicidin; LL37; magainin; maximum H5; melittin; moricin; prophenin; protegrin; and or tachyplesins.

According to a particular embodiment, the antibiotic is a non-absorbable antibiotic.

Non-antibiotic agents which alter the circadian rhythmicity of microbes of the gut microbiome of the subject are also contemplated by the present inventors.

In one embodiment, the agent which alters the circadian rhythmicity of microbes of the gut microbiome of the subject is a bacterial population—i.e. a probiotic composition.

Preferably, the bacterial populations of this embodiment of the present invention are capable of increasing the growth and/or colonization of at least one bacterial strain/species.

An exemplary bacterial population according to this embodiment of the present invention comprises polyamine producing bacteria (for example Corynebacterium glutamicum or Escherichia coli). Other examples of polyamine producing bacteria are disclosed in Schneider et al., Appl Microbiol Biotechnol. 2011 July; 91(1):17-30. doi: 10.1007/s00253-011-3252-0. Epub 2011 May 7, the contents of which is incorporated herein by reference.

According to a particular embodiment, at least 10% of the bacteria of the bacterial populations of this aspect of the present invention are polyamine producing bacteria, at least 10% of the bacteria of the bacterial populations of this aspect of the present invention are polyamine producing bacteria, at least 20% of the bacteria of the bacterial populations of this aspect of the present invention are polyamine producing bacteria, at least 30% of the bacteria of the bacterial populations of this aspect of the present invention are polyamine producing bacteria, at least 40% of the bacteria of the bacterial populations of this aspect of the present invention are polyamine producing bacteria, at least 50% of the bacteria of the bacterial populations of this aspect of the present invention are polyamine producing bacteria, at least 60% of the bacteria of the bacterial populations of this aspect of the present invention are polyamine producing bacteria, at least 70% of the bacteria of the bacterial populations of this aspect of the present invention are polyamine producing bacteria, at least 80% of the bacteria of the bacterial populations of this aspect of the present invention are polyamine producing bacteria or even at least 90% of the bacteria of the bacterial populations of this aspect of the present invention are polyamine producing bacteria.

The probiotic compositions may comprise more than 100 bacterial species, more than 90 bacterial species, more than 80 bacterial species, more than 70 species strains, more than 60 bacterial species, more than 50 bacterial species, more than 40 bacterial species, more than 30 bacterial species, more than 20 bacterial species, more than 10 bacterial species, more than 9 bacterial species, more than 8 bacterial species, more than 7 bacterial species, more than 6 bacterial species, more than 5 bacterial species, more than 4 bacterial species, more than 3 bacterial species, more than 2 bacterial species or even 1 bacterial species.

In one embodiment, the probiotic composition is a fecal transplant derived from a healthy subject.

The present inventors contemplate any formulation for the microbial compositions so long as the bacterial population within is capable of propagating when administered to the subject.

The compositions of the present invention may be formulated as a food supplement, an enema, a tablet, a capsule or a syringe

The compositions of the invention can be formulated as a slurry, saline or buffered suspensions (e.g., for an enema, suspended in a buffer or a saline), in a drink (e.g., a milk, yoghurt, a shake, a flavoured drink or equivalent) for oral delivery, and the like.

In alternative embodiments, compositions of the invention can be formulated as an enema product, a spray dried product, reconstituted enema, a small capsule product, a small capsule product suitable for administration to children, a bulb syringe, a bulb syringe suitable for a home enema with a saline addition, a powder product, a powder product in oxygen deprived sachets, a powder product in oxygen deprived sachets that can be added to, for example, a bulb syringe or enema. or a spray dried product in a device that can be attached to a container with an appropriate carrier medium such as yoghurt or milk and that can be directly incorporated and given as a dosing for example for children.

In one embodiment, compositionsof the invention can be delivered directly in a carrier medium via a screw-top lid wherein the bacterial material is suspended in the lid and released on twisting the lid straight into the carrier medium.

In alternative embodiments methods of delivery of compositions of the inv ration include use of bacterial slurries into the bowel, via an enema suspended in saline or a buffer, via a small bowel infusion via a nasoduodenal tube, via a gastrostomy, or by using a colonoscope.

According to still another embodiment, the microbial composition of any of the aspects of the present invention is devoid (or comprises only trace quantities) of fecal material (e.g., fiber).

The probiotic bacteria may be in any suitable form, for example in a powdered dry form. In addition, the probiotic microorganism may have undergone processing in order for it to increase its survival. For example, the microorganism may be coated or encapsulated in a polysaccharide, fat, starch, protein or in a sugar matrix. Standard encapsulation techniques known in the art can be used. For example, techniques discussed in U.S. Pat. No. 6,190,591, which is hereby incorporated by reference in its entirety, may be used.

According to a particular embodiment, the probiotic microorganism composition is formulated in a food product, functional food or nutraceutical.

In some embodiments, a food product, functional food or nutraceutical is or comprises a dairy product. In some embodiments, a dairy product is or comprises a yogurt product. In some embodiments, a dairy product is or comprises a milk product. In some embodiments, a dairy product is or comprises a cheese product. In some embodiments, a food product, functional food or nutraceutical is or comprises a juice or other product derived from fruit. In some embodiments, a food product, functional food or nutraceutical is or comprises a product derived from vegetables. In some embodiments, a food product, functional food or nutraceutical is or comprises a grain product, including but not limited to cereal, crackers, bread, and/or oatmeal. In some embodiments, a food product, functional food or nutraceutical is or comprises a rice product. In some embodiments, a food product, functional food or nutraceutical is or comprises a meat product.

Prior to administration, the subject may be pretreated with an agent which reduces the number of naturally occurring microbes in the microbiome (e.g. by antibiotic treatment). According to a particular embodiment, the treatment significantly eliminates the naturally occurring gut microflora by at least 20%, 30% 40%, 50%, 60%, 70%, 80% or even 90%.

In still another embodiment, the agent which alters the circadian rhythmicity of microbes of the gut microbiome of the subject is a metabolite.

As used herein, a “metabolite” is an intermediate or product of metabolism. The term metabolite is generally restricted to small molecules and does not include polymeric compounds such as DNA or proteins. A metabolite may serve as a substrate for an enzyme of a metabolic pathway, an intermediate of such a pathway or the product obtained by the metabolic pathway.

According to a particular embodiment, the metabolite is one that alters the composition or function of the microbiome.

In preferred embodiments, metabolites include but are not limited to sugars, organic acids, amino acids, fatty acids, hormones, vitamins, oligopeptides (less than about 100 amino acids in length), as well as ionic fragments thereof. Cells can also be lysed in order to measure cellular products present within the cell. In particular, the metabolites are less than about 3000 Daltons in molecular weight, and more particularly from about 50 to about 3000 Daltons.

The metabolite of this aspect of the present invention may be a primary metabolite (i.e. essential to the microbe for growth) or a secondary metabolite (one that does not play a role in growth, development or reproduction, and is formed during the end or near the stationary phase of growth.

Representative examples of metabolic pathways in which the metabolites of the present invention are involved include, without limitation, citric acid cycle, respiratory chain, photosynthesis, photorespiration, glycolysis, gluconeogenesis, hexose monophosphate pathway, oxidative pentose phosphate pathway, production and β-oxidation of fatty acids, urea cycle, amino acid biosynthesis pathways, protein degradation pathways such as proteasomal degradation, amino acid degrading pathways, biosynthesis or degradation of: lipids, polyketides (including, e.g., flavonoids and isoflavonoids), isoprenoids (including, e.g., terpenes, sterols, steroids, carotenoids, xanthophylls), carbohydrates, phenylpropanoids and derivatives, alkaloids, benzenoids, indoles, indole-sulfur compounds, porphyrines, anthocyans, hormones, vitamins, cofactors such as prosthetic groups or electron carriers, lignin, glucosinolates, purines, pyrimidines, nucleosides, nucleotides and related molecules such as tRNAs, microRNAs (miRNA) or mRNAs.

According to a particular embodiment, the metabolite is a polyamine. Examples of polyamines contemplated by the present invention include, but are not limited to putrescine, cadaverine, spermidine, and spermine.

According to one embodiment, the metabolite is selected from the group consisting of taurine, pinitol, sebacate, undecanedioate, dodencanedioate, homoserine, taurodeoxycholate, chenodeoxycholate, tryptamine, glutarate, ethylmalonate, histamine, spermidine, putrescine, cadaverine, spermine, AMP, GABA, N-acetyltryptophan, pipecolic acid and N-acetylproline.

Other agents which alter the circadian rhythmicity of microbes of the gut microbiome include a food or beverage which is rich in polyamines.

Fruits (like oranges), fruit juices (orange juice and grapefruit juice), sauerkraut, cheddar cheeses, cod roe, soy sauce, and soy miso are known to be high in putrescine. Spermidine content is high in dry soy bean, chicken liver, green peas, corn, shell fish, and blue cheese. A high content of spermine may be found in most of the meat products (like sausages, pork, chicken, and turkey), some vegetables (like pumpkin), and cheese.

Another way to alter the circadian rhythmicity of microbes of the gut is by eating a polyamine deficient diet and/or a fat-rich diet.

In one embodiment, the agent which alters the circadian rhythmicity of microbes of the gut is determined on an individual basis by analysis of the microbes of the subject's microbiome. Analysis of the microbiome is described in detail herein below.

The present inventors also propose that agents which alter the circadian rhythmicity of microbes of the gut microbiome of the subject can be used to reduce the liver toxicity of a liver-damaging agent.

Thus, according to another aspect of the present invention there is provided a method of reducing the liver toxicity of a liver-damaging agent in a subject comprising administering to the subject:

(i) the liver-damaging agent; and

(ii) an agent which alters the circadian rhythmicity of microbes of the gut microbiome of the subject, thereby reducing the liver toxicity of the liver-damaging agent.

The liver-damaging agent of this aspect of the present invention may be pharmaceutical agents or diagnostic agents.

In one embodiment, the liver damaging agent is not an alcoholic beverage.

In another embodiment, the liver damaging agent is an alcoholic beverage.

Examples of pharmaceutical agents known to cause liver toxicity include but are not limited to acetaminophen, salicylates, isoniazid, Amiodarone, Methotrexate and Nitrofurantoin.

Specifically, pharmaceutical agents that cause acute dose dependent liver damage include but are not limited to salicylates and acetaminophen.

Pharmaceutical agents that cause acute dose independent liver damage include but are not limited to acebutolol, labetalol, quinine, ethionamide, indomethacin, probenecid, diltiazem, phenelzine, phenylbutazone, cimetidine, naproxen, tricyclic antidepressants, maprotiline, maprotiline, allopurinol, enflurane, halothane, isoniazid, pyrazinamide, para-aminosalicylic acid, phenindione, phenytoin, dantrolene, valproic acid, atenolol, metoprolol, sulfonamides, ibuprofen, ketoconazole, quinidine, ethambutol, Phenobarbital, piroxicam, diclofenac, penicillins, verapamil, carbamazepine, mianserin and sulindac.

Pharmaceutical agents that may cause acute fatty infiltration of the liver include, but are not limited to adrenocortical steroids, phenothiazines, sulfonamides, antithyroid drugs, phenytoin, tetracyclines, isoniazid, salicylates, valproic acid and methotrexate.

Pharmaceutical agents that may cause cholestatic jaundice include but are not limited to actinomycin D, chlorpropamide, erythromycin, amoxicillin/clavulanate, cloxacillin flecainide, azathioprine, cyclophosphamide, flurazepam, captopril, cyclosporine, flutamide, carbamazepine, danazol, glyburide, carbimazole, diazepam, gold, cephalosporins, disopyramide, griseofulvin, chlordiazepoxide, enalapril, haloperidol, ketoconazole, norethandrolone, sulfonamides, mercaptopurine, oral contraceptives, tamoxifen, methyltestosterone, oxacillin, thiabendazole, nifedipine, penicillamine, tolbutamide, nitrofurantoin, phenothiazines, tricyclic antidepressants, phenytoin troleandomycin, anti-inflammatory drugs, propoxyphene and verapamil.

Pharmaceutical agents that may cause liver granulomas (chronic inflammatory nodules) include but are not limited to allopurinol, gold, phenytoin, aspirin, hydralazine, procainamide, carbamazepine, isoniazid, quinidine, chlorpromazine, nitrofurantoin, sulfonamides, diltiazem, penicillin, tolbutamide and disopyramide.

Pharmaceutical agents that may cause active chronic hepatitis include but are not limited to acetaminophen, dantrolene, methyldopa, isoniazid and nitrofurantoin.

Pharmaceutical agents that may cause liver cirrhosis or fibrosis (scarring) include but are not limited to methotrexate, terbinafine HCl (Lamisil, Sporanox) and nicotinic acid.

Pharmaceutical agents that may cause chronic cholestasis (resembling primary biliary cirrhosis) include but are not limited to chlorpromazine/valproic acid (combination), imipramine, thiabendazole, phenothiazines, tolbutamide, chlorpropamide/erythro-mycin (combination) and phenytoin.

Pharmaceutical agents that may cause liver tumors (benign and malignant) include, but are not limited to anabolic steroids, oral contraceptives, thorotrast, danazol and testosterone.

Pharmaceutical agents that may cause damage to liver blood vessels include, but are not limited to adriamycin, dacarbazine, thioquanine, anabolic steroids, mercaptopurine, vincristine, azathioprine, methotrexate, vitamin A (excessive doses), carmustine, mitomycin, cyclophosphamide/cyclo-sporine (combination) and oral contraceptives.

Agents which alter the circadian rhythmicity of microbes of the gut microbiome of the subject are described herein above.

For any of the aspects described herein above, administering comprises any means of administering an effective (e.g., therapeutically effective) or otherwise desirable amount of a composition to an individual. In some embodiments, administering a composition comprises administration by any route, including for example parenteral and non-parenteral routes of administration. Parenteral routes include, e.g., intraarterial, intracerebroventricular, intracranial, intramuscular, intraperitoneal, intrapleural, intraportal, intraspinal, intrathecal, intravenous, subcutaneous, or other routes of injection. Non-parenteral routes include, e.g., buccal, nasal, ocular, oral, pulmonary, rectal, transdermal, or vaginal.

According to a particular embodiment, the administration is by rectal administration (e.g. enema).

Administration may also be by continuous infusion, local administration, sustained release from implants (gels, membranes or the like), and/or intravenous injection.

Particular doses or amounts to be administered in accordance with the present invention may vary, for example, depending on the nature and/or extent of the desired outcome, on particulars of route and/or timing of administration, and/or on one or more characteristics (e.g., weight, age, personal history, genetic characteristic, lifestyle parameter, severity of disease, etc., or combinations thereof). Such doses or amounts can be determined by those of ordinary skill. In some embodiments, an appropriate dose or amount is determined in accordance with standard clinical techniques. Alternatively or additionally, in some embodiments, an appropriate dose or amount is determined through use of one or more in vitro or in vivo assays to help identify desirable or optimal dosage ranges or amounts to be administered.

In some particular embodiments, appropriate doses or amounts to be administered may be extrapolated from dose-response curves derived from in vitro or animal model test systems. The effective dose or amount to be administered for a particular individual can be varied (e.g., increased or decreased) over time, depending on the needs of the individual. In some embodiments, where bacteria are administered, an appropriate dosage comprises at least about 100, 200, 300, 400, 500, 600, 700, 800, 900, 1000 or more bacterial cells. In some embodiments, the present invention encompasses the recognition that greater benefit may be achieved by providing numbers of bacterial cells greater than about 1000 or more (e.g., than about 1500, 2000, 2500, 3000, 35000, 4000, 4500, 5000, 5500, 6000, 7000, 8000, 9000, 10,000, 15,000, 20,000, 25,000, 30,000, 40,000, 50,000, 75,000, 100,000, 200,000, 300,000, 400,000, 500,000, 600,000, 700,000, 800,000, 900,000, 1×10⁶, 2×10⁶, 3 ×10⁶, 4 ×10⁶, 5 ×10⁶, 6 ×10⁶, 7 ×10⁶, 8 ×10⁶, 9 ×10⁶, 1 ×10⁷, 1 ×10⁸, 1 ×10⁹, 1 ×10¹⁰, 1 ×10¹¹, 1 ×10¹², 1 ×10¹³ or more bacteria.

In the context of a combination therapy, combination therapy agents may be administered by the same route of administration (e.g. rectal, oral, enteral, etc.) or alternatively, the agents for use in combination therapy may each be administered by a different route of administration.

If administered via the same route of administration, the present inventors further contemplate that both of the active agents are co-formulated in one composition. Alternatively, the present inventors further contemplate that the active agents are formulated in separate compositions.

The agent which alters the circadian rhythmicity of microbes of the gut microbiome of the subject can be administered immediately prior to (or after) the agent which is used to treat the liver disease (or the liver damaging agent), on the same day as, one day before (or after), one week before (or after), one month before (or after), or two months before (or after) the agent which is used to treat the disease (e.g. liver disease), and the like.

The agents which alter the circadian rhythmicity of microbes of the gut microbiome and the agents which are used to treat the liver disease (or the liver damaging agent) can be administered concomitantly, that is, where the administering for each of these reagents can occur at time intervals that partially or fully overlap each other. The agents can be administered during time intervals that do not overlap each other. For example, the first agent can be administered within the time frame of t=0 to 1 hours, while the second agent can be administered within the time frame of t=1 to 2 hours. Also, the first agent can be administered within the time frame of t=0 to 1 hours, while the second agent can be administered somewhere within the time frame of t=2-3 hours, t=3-4 hours, t=4-5 hours, t=5-6 hours, t=6-7 hours, t=7-8 hours, t=8-9 hours, t=9-10 hours, and the like. Moreover, the second agent can be administered somewhere in the time frame of t=minus 2-3 hours, t=minus 3-4 hours, t=minus 4-5 hours, t=5-6 minus hours, t=minus 6-7 hours, t=minus 7-8 hours, t=minus 8-9 hours, t=minus 9-10 hours.

The agents which alter the circadian rhythmicity of microbes of the gut microbiome and the agents which are used to treat the liver disease (or the liver damaging agent) (i.e. second reagent) are typically provided in combined amounts to achieve therapeutic, prophylactic effectiveness. This amount will evidently depend upon the particular agent selected for use, the nature and number of the other treatment modality, the condition(s) to be treated, prevented and/or palliated, the species, age, sex, weight, health and prognosis of the subject, the mode of administration, effectiveness of targeting, residence time, mode of clearance, type and severity of side effects of the pharmaceutical composition and upon many other factors which will be evident to those of skill in the art.

Due to the effect the agent which alters the circadian rhythmicity of microbes of the gut microbiome has on the liver, the agent which is typically used to treat the disease (e.g. liver disease) is used at a level between 10% of its normal minimum therapeutic dose and 100% of its maximum normal therapeutic dose. More preferably this range will be 25% of its normal minimum dose to 90% of its normal maximum dose.

In one preferred embodiment, the amount of the agent which is used to treat the liver disease (or the liver damaging agent) is below the minimum dose required for therapeutic or prophylactic effectiveness when used as a single therapy (e.g. 10-99%, preferably 25 to 75% of that minimum dose). This allows for reduction of the side effects caused by the agent used to treat the liver disease (or the liver damaging agent) but the therapy is rendered effective because in combination with the agent which alters the circadian rhythmicity of microbes of the gut microbiome, the combinations are effective overall.

Since the present inventors have shown that the circadian rhythmicity of bacteria of the gut microbiome may affect the toxicity of a pharmaceutical agent, the present inventors further propose analysis of the circadian rhythmicity of at least one component of the gut microbiome of a subject in order to determine a dose or treatment regimen.

Thus, according to yet another aspect of the present invention there is provided a method of selecting a dose or treatment regimen of a pharmaceutical agent for a subject comprising:

(a) analyzing the circadian rhythmicity of at least one component of the gut microbiome of the subject; and

(b) selecting the dose or treatment regimen according to the circadian rhythmicity.

The phrase “at least one component” refers to a microbial component—i.e. at least one bacteria, fungi, virus etc.

Measuring a level or presence of a microbe may be effected by analyzing for the presence of microbial component or a microbial by-product. Thus, for example the level or presence of a microbe may be effected by measuring the level of a DNA sequence. In some embodiments, the level or presence of a microbe may be effected by measuring 16S rRNA gene sequences or 18S rRNA gene sequences. In other embodiments, the level or presence of a microbe may be effected by measuring RNA transcripts. In still other embodiments the level or presence of a microbe may be effected by measuring proteins. In still other embodiments, the level or presence of a microbe may be effected by measuring metabolites.

The present embodiments encompass the recognition that microbial signatures can be relied upon as proxy for microbiome composition and/or activity. Microbial signatures comprise data points that are indicators of microbiome composition and/or activity. Thus, according to the present invention, changes in microbiomes can be detected and/or analyzed through detection of one or more features of microbial signatures.

In some embodiments, a microbial signature includes information relating to absolute amount of one or more types of microbes, and/or products thereof. In some embodiments, a microbial signature includes information relating to relative amounts of five, ten, twenty or more types of microbes and/or products thereof.

Examples of microbial products include, but are not limited to mRNAs, polypeptides, carbohydrates and metabolites.

In some embodiments, a microbial signature includes information relating to presence, level, and/or activity of at least ten types of microbes. In some embodiments, a microbial signature includes information relating to presence, level, and/or activity of between 5 and 100 types of microbes. In some embodiments, a microbial signature includes information relating to presence, level, and/or activity of between 100 and 1000 or more types of microbes. In some embodiments, a microbial signature includes information relating to presence, level, and/or activity of substantially all types of bacteria within the microbiome. In some embodiments, a microbial signature includes information relating to presence, level, and/or activity of substantially all types of microbes within the microbiome.

In some embodiments, a microbial signature includes information relating to presence, level, and/or activity of metabolites of at least ten types of microbes. In some embodiments, a microbial signature includes information relating to presence, level, and/or activity of metabolites of between 5 and 100 types of microbes. In some embodiments, a microbial signature includes information relating to presence, level, and/or activity of metabolites of between 100 and 1000 or more types of microbes. In some embodiments, a microbial signature includes information relating to presence, level, and/or activity of substantially metabolites of all types of bacteria within the microbiome. In some embodiments, a microbial signature includes information relating to presence, level, and/or activity of metabolites of substantially all types of microbes within the microbiome.

According to this aspect of the present invention the microbiome signature includes a presence or level of at least one, at least 10, at least 20, at least 50, at least 100, at least 200, at least 300, at least 400, at least 500, at least 600, at least 700, at least 800, at least 900, at least 1000, at least 1200, at least 1500 or all the species of microbes of the microbiome.

In some embodiments, a microbiome signature comprises a level or set of levels of at least one, or at least five, or at least ten or more types of microbes (e.g. bacteria) or components or by-products thereof. In some embodiments, a microbial signature comprises a level or set of levels of at least one or at least five or at least ten or more DNA sequences. In some embodiments, a microbial signature comprises a level or set of levels of ten or more 16S rRNA gene sequences. In some embodiments, a microbial signature comprises a level or set of levels of 18S rRNA gene sequences. In some embodiments, a microbial signature comprises a level or set of levels of at least five or at least ten or more RNA transcripts. In some embodiments, a microbial signature comprises a level or set of levels of at least five or at least ten or more proteins. In some embodiments, a microbial signature comprises a level or set of levels of at least one or at least five or at least ten or more metabolites.

Obtaining a Microbiome Sample

In order to analyze the microbiome, samples are taken from a subject.

The subject is typically a mammalian subject—e.g. human subject.

In order to analyze circadian rhythmicity of a component of the gut microbiome, a sample of the microbiome is analyzed. In one embodiment, the sample is a fecal sample.

Analysis is typically effected at least once a day, every 12 hours, every 6 hours or any other time period. Preferably, the time period between each analysis is identical. Preferably samples are taken at a fixed time in the day.

Obtaining chromosomal (genomic) DNA from microbiomes may be effected using conventional techniques, for example as disclosed in Sambrook and. Russell, Molecular Cloning: A Laboratory Manual, cited supra. In some cases, particularly if small amounts of DNA are employed in a particular step, it is advantageous to provide carrier DNA, e.g. unrelated circular synthetic double-stranded DNA, to be mixed and used with the sample DNA whenever only small amounts of sample DNA are available and there is danger of losses through nonspecific binding, e.g, to container walls and the like.

In one embodiment. long fragments of chromosomal DNA are obtained. Cells are lysed and the intact nuclei may be pelleted with a gentle centrifugation step. The genomic DNA is then released (e.g. through proteinase K and RNase digestion, for several hours (e.g. 1-5 hours)). The material can be treated to lower the concentration of remaining cellular waste, e.g., by dialysis for a period of time (i.e., from 2-16 hours) and/or dilution. Since such methods need not employ many disruptive processes (such as ethanol precipitation, centrifugation, and vortexing), the genomic nucleic acid remains largely intact, yielding a majority of fragments that have lengths in excess of 150 kilobases. In some embodiments, the fragments are from about o about 750 kilobases in lengths. In further embodiments, the fragments are from about 150 to about 600, about 200 to about 500, about 250 to about 400, and about 300 to about 350 kilobases in length.

Optionally, the target genomic DNA is then fractionated or fragmented to a desired size by conventional techniques including enzymatic digestion, shearing, or sonication, with the latter two finding particular use in the present invention.

Fragment sizes of the target nucleic acid can vary depending on the source target nucleic acid, and the library construction methods used, but for standard whole-genome sequencing such fragments may range from 50 to 600 nucleotides in length. In another embodiment, the fragments are 300 to 600 or 200 to 2000 nucleotides in length. In yet another embodiment, the fragments are 10-1.00, 50-100, 50-300, 100-200, 200-300, 50-400, 100-400, 200-400, 300-400, 400-500, 400-600, 500-600 50-1000, 100-1000, 200-1000, 300-1000, 400-1000, 500-1000, 600-1000, 700-1000, 700-900, 700-800, 800-1000, 900-1000, 1500-2000, 1750-2000, and 50-2000 nucleotides in length. Longer fragments are also contemplated.

In a further embodiment, fragments of a particular size or in a particular range of sizes are isolated. Such methods are well known in the art. For example, gel fractionation can be used to produce a population of fragments of a particular size within a range of basepairs, for example for 500 base pairs+50 base pairs,

In many cases, enzymatic digestion of extracted DNA is not required because shear forces created during lysis and extraction will generate fragments in the desired range. In a further embodiment, shorter fragments (1-5 kb) can be generated by enzymatic fragmentation using restriction endonucleases.

Quantifying Microbial Levels:

It will be appreciated that determining the abundance of microbes may be affected by taking into account any feature of the microbiome. Thus, the abundance of microbes may be affected by taking into account the abundance at different phylogenetic levels; at the level of gene abundance; gene metabolic pathway abundances; sub-species strain identification; SNPs and insertions and deletions in specific bacterial regions; growth rates of bacteria, the diversity of the microbes of the microbiome, as further described herein below.

In some embodiments, determining a level or set of levels of one or more types of microbes or components or products thereof comprises determining a level or set of levels of one or more DNA sequences. In some embodiments, one or more DNA sequences comprises any DNA sequence that can be used to differentiate between different microbial types. In certain embodiments, one or more DNA sequences comprises 16S rRNA gene sequences. In certain embodiments, one or more DNA sequences comprises 18S rRNA gene sequences. In some embodiments, 1, 2, 3, 4, 5, 10, 15, 20, 25, 50, 100, 1,000, 5,000 or more sequences are amplified.

16S and 18S rRNA gene sequences encode small subunit components of prokaryotic and eukaryotic ribosomes respectively. rRNA genes are particularly useful in distinguishing between types of microbes because, although sequences of these genes differ between microbial species, the genes have highly conserved regions for primer binding. This specificity between conserved primer binding regions allows the rRNA genes of many different types of microbes to be amplified with a single set of primers and then to be distinguished by amplified sequences.

In some embodiments, a microbiota sample (e.g. fecal sample) is directly assayed for a level or set of levels of one or more DNA sequences. In some embodiments, DNA is isolated from a microbiota sample and isolated DNA is assayed for a level or set of levels of one or more DNA sequences. Methods of isolating microbial DNA are well known in the art. Examples include but are not limited to phenol-chloroform extraction and a wide variety of commercially available kits, including QIAamp DNA Stool Mini Kit (Qiagen, Valencia, Calif.).

In some embodiments, a level or set of levels of one or more DNA sequences is determined by amplifying DNA sequences using PCR (e.g., standard PCR, semi-quantitative, or quantitative PCR). In some embodiments, a level or set of levels of one or more DNA sequences is determined by amplifying DNA sequences using quantitative PCR. These and other basic DNA amplification procedures are well known to practitioners in the art and are described in Ausebel et al. (Ausubel F M, Brent R, Kingston R E, Moore D, Seidman J G, Smith J A, Struhl K (eds). 1998. Current Protocols in Molecular Biology. Wiley: New York).

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences.

In some embodiments, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

In some embodiments, the abundance of a microbe is determined by DNA sequencing.

Methods for sequence determination are generally known to the person skilled in the art. Preferred sequencing methods are next generation sequencing methods or parallel high throughput sequencing methods. For example, a bacterial genomic sequence may be obtained by using Massively Parallel Signature Sequencing (MPSS). An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLiD technology, which employs sequencing by ligation. This method is based on the use of a pool of all possible oligonucleotides of a fixed length, which are labeled according to the sequenced position. Such oligonucleotides are annealed and ligated.

Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods. The present invention also envisages further developments of these techniques, e.g. further improvements of the accuracy of the sequence determination, or the time needed for the determination of the genomic sequence of an organism etc.

According to one embodiment, the sequencing method comprises deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

In some embodiments, determining a level or set of levels of one or more types of microbes comprises determining a level or set of levels of one or more microbial RNA molecules (e.g., transcripts). Methods of quantifying levels of RNA transcripts are well known in the art and include but are not limited to northern analysis, semi-quantitative reverse transcriptase PCR, quantitative reverse transcriptase PCR, and microarray analysis.

In some embodiments, determining a level or set of levels of one or more types of microbes comprises determining a level or set of levels of one or more microbial polypeptides. Methods of quantifying polypeptide levels are well known in the art and include but are not limited to Western analysis and mass spectrometry.

As mentioned herein above, as well as (or instead of) analyzing the abundance of microbes, the present invention also contemplates analyzing the level of microbial products.

Examples of microbial products include, but are not limited to mRNAs, polypeptides, carbohydrates and metabolites.

In some embodiments, levels of metabolites are determined by mass spectrometry. In some embodiments, levels of metabolites are determined by nuclear magnetic resonance spectroscopy, as further described herein below. In some embodiments, levels of metabolites are determined by enzyme-linked immunosorbent assay (ELISA). In some embodiments, levels of metabolites are determined by colorimetry. In some embodiments, levels of metabolites are determined by spectrophotometry.

As mentioned, as well as (or instead of) determining the abundance of the specified microbes at various times of the day, the present inventors also contemplate analyzing the growth dynamics of the microbes of the microbiome at various times of the day.

The term “growth dynamics” refers to the growth phase of a bacterium (e.g. lag phase, stationary phase, exponential growth, death phase) and to the growth rate itself.

During lag phase, bacteria adapt themselves to growth conditions. It is the period where the individual bacteria are maturing and not yet able to divide. During the lag phase of the bacterial growth cycle, synthesis of RNA, enzymes and other molecules occurs.

The log phase (sometimes called the logarithmic phase or the exponential phase) is a period characterized by cell doubling. The number of new bacteria appearing per unit time is proportional to the present population. If growth is not limited, doubling will continue at a constant rate so both the number of cells and the rate of population increase doubles with each consecutive time period. For this type of exponential growth, plotting the natural logarithm of cell number against time produces a straight line. The slope of this line is the specific growth rate of the organism, which is a measure of the number of divisions per cell per unit time. The actual rate of this growth depends upon the growth conditions, which affect the frequency of cell division events and the probability of both daughter cells surviving. Exponential growth cannot continue indefinitely, however, because the medium is soon depleted of nutrients and enriched with wastes.

The stationary phase is often due to a growth-limiting factor such as the depletion of an essential nutrient, and/or the formation of an inhibitory product such as an organic acid. Stationary phase results from a situation in which growth rate and death rate are equal. The number of new cells created is limited by the growth factor and as a result the rate of cell growth matches the rate of cell death.

At death phase, (Decline phase) bacteria die. This could be due to lack of nutrients, a temperature which is too high or low, or the wrong living conditions.

According to a particular embodiment, measuring the growth dynamics is effected by:

(a) sequencing DNA fragments of the microbial species or microbial strain of a microbiome to obtain a plurality of nucleic acid sequencing data;

(b) aligning the plurality of nucleic acid sequence data to at least one reference sequence, the reference sequence being of a genome of the microbial species of microbial strain; and

(c) analyzing the frequency of at least one nucleotide positioned at the origin of replication of the genome and the frequency of at least one nucleotide positioned at the terminus of the genome, wherein the ratio of the frequencies is indicative of the growth dynamics of the particular microbe.

The sequencing step of this embodiment may be carried out using any method known in the art, as further described herein above.

Once the plurality of sequencing data has been obtained, the next step comprises aligning the plurality of nucleic acid sequence data to at least one reference sequence, the reference sequence being of the genome of the bacterium being analyzed.

The term “aligning to a reference sequence” as used herein refers to the comparison of nucleic acid fragment read information and their arrangement with an already existing genomic or sub-genomic sequence, preferably followed by a placement of the sequence read stretches within a scaffold provided by the preexisting genomic or sub-genomic sequence.

The “reference sequence” as used herein may be any suitable preexisting sequence covering the stretch, which is identical or similar to the newly obtained sequence data or nucleic acid fragment reads.

Bacterial genome sequences (reference sequences) may be derived from NCBI's microbial genome project database and other databases disclosed in Qin, J. et al. A metagenome-wide association study of gut microbiota in type 2 diabetes. Nature 490, 55-60 (2012), incorporated herein by reference and Nielsen, H. B. et al. Identification and assembly of genomes and genetic elements in complex metagenomic samples without using reference genomes. Nat. Biotechnol. (2014). doi:10.1038/nbt.2939, incorporated herein by reference.

Further details may be derived from McNeil L K et al., The National Microbial Pathogen Database Resource (NMPDR): a genomics platform based on subsystem annotation, Nucleic Acids Res., 2007; 35 (Database issue): D347-53, which is incorporated herein by reference in its entirety.

The reference sequence may be essentially complete or comprise sub-portions of an essentially complete bacterial genome as defined below.

The term “essentially complete” as used herein refers to the presence of sequence information on all portions of the genome present in nature. For example, the genome sequence may comprise redundant sequences, repeats, telomeric sequences etc. For example, about 99%, 98%, 97%, 95%, 90%, 85%, 80%, or 75% of the genome sequence may be comprised in an essentially complete genome. In further embodiments, the reference sequence may not comprise certain sequence elements, such as repeats, telomeric sequences, transposon sequences, redundant sequences etc.

A “sub-portion” of an essentially complete genome may, for example, be any percentage of the entire genomic sequence, e.g. 10%, 20%, 30%, 40%, 60%, 65%, 70%, 75% etc. or any value in between. A sub-portion may also be a single chromosome sequence, a chromosomal arm, a combination of more than one chromosome, a haploid chromosomal set etc.

In a further preferred embodiment of the present invention the reference sequence as mentioned herein above may be selected from a group or taxon, which is phylogenetically related to the organism, whose nucleic acid data is to be assembled. Generally, a phylogenetically related organism may have an overall genomic identity of at least about 50%, 60%, 70%, 80%, 90% or 95%. A phylogenetically related organism may, for example, be a C. glutamticum in reference to E. coli sequence reads to be analyzed, or vice versa etc. In further embodiments, a reference sequence derived from a phylogenetically related organism may comprise a sub-portion of the entire genomic sequence, e.g. comprise certain chromosomes, chromosome combinations, chromosome arms, sections of the genome etc. as defined herein above.

The alignment to a reference sequence according to step (b) of the method according to the present invention may, in preferred embodiments, be carried out with or based on a suitable reference alignment algorithm. Preferred examples of such algorithms include the algorithms BFAST, ELAND, GenomeMapper, GMAP, MAQ, MOSAIK, PASS, SeqMap, SHRiMP, SOAP, SSAHA, or CLD. Particularly preferred is the use of the algorithms Bowtie or BWA. Further envisaged is the combination of one or more of these algorithms. For example, a reference alignment may first be carried out with one of the mentioned algorithms, followed by a repetition by a different of these algorithms. Results of both procedures may be compared and, where appropriate, combined. It is, in general, preferred to use contiguous nucleotide sequences showing a minimum number of non-matching reads or non-aligned reads.

Details and ways of implementing these algorithms would be known to the person skilled in the art, or can be derived from suitable literature sources, e.g. from Bao et al., Journal of Human Genetics, 28 Apr. 2011, p. 1-9, which is incorporated herein by reference in its entirety. The present invention further envisages the use of optimized or further developed versions of these algorithms, or of reference alignment algorithms following a different scheme or algorithmic logic including not yet available algorithms, as long as the principle purpose of an alignment to a reference sequence as described herein is fulfilled.

Following alignment, the method continues by analyzing the frequency of at least one nucleotide positioned at the origin of replication of the genome and the frequency of at least one nucleotide positioned at the terminus of the genome, wherein the ratio of the frequencies is indicative of the growth dynamics of the bacterium.

As used herein the “origin of replication” refers to a particular sequence in a genome at which replication is initiated. The specific structure of the origin of replication varies somewhat from species to species, but all share some common characteristics such as high AT content (adenine and thymine). The origin of replication binds the pre-replication complex, a protein complex that recognizes, unwinds, and begins to copy DNA.

Most bacteria have a single circular molecule of DNA, and typically only a single origin of replication per circular chromosome.

The terminus of the genome is typically positioned approximately opposite the origin or replication on the circular bacterial genome.

The terminus region contains several DNA replication terminator sites, or “Ter” sites.

Analyzing the frequency of at least one nucleotide positioned at the origin of replication and the frequency of at least one nucleotide positioned at the terminus of the genome may be effected by analyzing the coverage pattern of the reads at these positions.

In another embodiment, the frequency of 20% of the nucleotides across the genome of the bacterium is analyzed, wherein at least one of the nucleotides which is analyzed is positioned at the origin of replication and at least one of the nucleotides is positioned at the terminus.

In another embodiment, the frequency of 30% of the nucleotides across the genome of the bacterium is analyzed, wherein at least one of the nucleotides which is analyzed is positioned at the origin of replication and at least one of the nucleotides is positioned at the terminus.

In another embodiment, the frequency of 40% of the nucleotides across the genome of the bacterium is analyzed, wherein at least one of the nucleotides which is analyzed is positioned at the origin of replication and at least one of the nucleotides is positioned at the terminus.

In another embodiment, the frequency of 50% of the nucleotides across the genome of the bacterium is analyzed, wherein at least one of the nucleotides which is analyzed is positioned at the origin of replication and at least one of the nucleotides is positioned at the terminus.

In another embodiment, the frequency of 60% of the nucleotides across the genome of the bacterium is analyzed, wherein at least one of the nucleotides which is analyzed is positioned at the origin of replication and at least one of the nucleotides is positioned at the terminus.

In another embodiment, the frequency of 70% of the nucleotides across the genome of the bacterium is analyzed, wherein at least one of the nucleotides which is analyzed is positioned at the origin of replication and at least one of the nucleotides is positioned at the terminus.

In another embodiment, the frequency of 80% of the nucleotides across the genome of the bacterium is analyzed, wherein at least one of the nucleotides which is analyzed is positioned at the origin of replication and at least one of the nucleotides is positioned at the terminus.

In another embodiment, the frequency of 90% of the nucleotides across the genome of the bacterium is analyzed, wherein at least one of the nucleotides which is analyzed is positioned at the origin of replication and at least one of the nucleotides is positioned at the terminus.

In another embodiment, the frequency of 95% of the nucleotides across the genome of the bacterium is analyzed, wherein at least one of the nucleotides which is analyzed is positioned at the origin of replication and at least one of the nucleotides is positioned at the terminus.

It will be appreciated that if the position of the origin of replication and the terminus are known, then the method of this aspect of the present invention may be carried out by analyzing the coverage (or frequency) at these positions only. However, if the position of the origin of replication and the terminus are not known, it is preferable that essentially all (or the majority) of the nucleotides across the genome are analyzed.

Optionally, the frequencies of the nucleotides may be displayed graphically as a function of their genomic location.

When the ratio of the frequency of a nucleotide at the origin of replication of the genome: frequency of the nucleotide at the terminus of the genome is about 2:1 or more, it is indicative of exponential growth of the bacterium.

Further, when the ratio of the frequency of a nucleotide at the origin of replication of the genome: frequency of a nucleotide at the terminus of the genome is about 1:1, it is indicative of stationary growth of the bacterium.

Bacteria can be classified as having statistically significantly similar growth dynamics at various points in the day if both are classified as stationary or both are classified as exponential.

According to one embodiment, if the frequency of the nucleotide at the origin of replication of the genome of the bacteria from the test microbiome at the first time point : frequency of the nucleotide at the terminus of the genome of the bacteria from the test microbiome at the first time point is about 2:1 or more and the frequency of the nucleotide at the origin of replication of the genome of the bacteria from the test microbiome at the second time point: frequency of the nucleotide at the terminus of the bacteria from the second time point is about 2:1 or more, then the two bacteria may be considered as having statistically significant similar growth dynamics.

Further, if the frequency of the nucleotide at the origin of replication of the genome of the bacteria from the first time point : frequency of the nucleotide at the terminus of the genome of the bacteria from the first time point is about 1:1 and the frequency of the nucleotide at the origin of replication of the genome of the bacteria from the second time point : frequency of the nucleotide at the terminus of the bacteria from the second time point is about 1:1, then the two bacteria may be considered as having statistically significant similar growth dynamics.

Other methods of analyzing bacterial growth dynamics are known in the art and include for example analysis of optical density of a bacterial inoculant over a period of time.

As mentioned, the dose or treatment regimen of a pharmaceutical agent is then selected according to the circadian rhythmicity of the microbes of the microbiome of the subject.

The present inventors contemplate determining the dose or treatment regimen of any pharmaceutical agent. In one embodiment, the pharmaceutical agent is one that is used to treat a liver disease. In another embodiment, the pharmaceutical agent is not an antibiotic. In another embodiment, the pharmaceutical agent is not used to treat a stomach or gut disorder.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Mice: C57B1/6 mice were purchased from Harlan and allowed to acclimatize to the animal facility environment for 2 weeks before used for experimentation. Germ-free C57B1/6 mice were born in the Weizmann Institute germ-free facility and routinely monitored for sterility. Mice lacking RegIIIγ (B6.129-Reg3g^{tm1.1Lvh}/J) were obtained from The Jackson Laboratory. In all experiments, age- and gender-matched mice were used. Mice were 8-9 weeks of age at the beginning of experiments. All mice were kept at a strict 24-hour light-dark cycle, with lights being turn on from 6 am to 6 pm. In timed feeding experiments, food access was limited to the dark phase or light phase where indicated. Polyamine-deficient diet was applied for one month. For antibiotic treatment, mice were given a combination of vancomycin (0.5 g/l), ampicillin (1 g/l), kanamycin (1 g/l), and metronidazole (1 g/l) in their drinking water for three weeks as previously described (Levy et al., 2015). Acetaminophen was administered intraperitoneally at a concentration of 500 mg/kg at either ZT0 or ZT12, and mice were analyzed 10 hours after injection. All antibiotics as well as acetaminophen were obtained from Sigma Aldrich.

Food intake and other metabolic parameters were measured using the PhenoMaster system (TSE-Systems, Bad Homburg, Germany), which consists of a combination of sensitive feeding sensors for automated measurement and a photobeam-based activity monitoring system detects oxygen and carbon dioxide consumption, and records ambulatory movements, including rearing and climbing, in each cage. All parameters were measured continuously and simultaneously. Mice were trained singly-housed in identical cages prior to data acquisition. All experimental procedures were approved by the local IACUC.

Scanning electron microscopy: Mice were perfused with fixative containing 2% glutaraldehyde and 3% PFA in 0.1M sodium cacodylate. Colonic samples were extensively washed from fecal matter and fixed for 24 hrs. Samples were rinsed three times in sodium cacodylate buffer and postfixed in 1% osmiumtetroxide for lhr, stained in 1% uranyl acetate for a further hour, then rinsed, dehydrated, and dried using critical point drying. Samples were then gold-coated and viewed in an ULTRA 55 FEG (ZEISS). For image quantification, the bacteria on randomly selected fields per sample were counted and averaged.

16S qPCR protocol for quantification of bacterial DNA: Colons were extensively cleaned from fecal material, and DNA was extracted using MoBio PowerSoil kit. DNA concentration was calculated using a standard curve of known DNA concentrations from E.coli K12. 16S qPCR using primers identifying different regions of the V6 16S gene was performed using Kappa SYBR fast mix, using the following primer sequences:

111-967F-PP:
(SEQ ID NO: 1)
CNACGCGAAGAACCTTANC
112-967F-UC3:
(SEQ ID NO: 2)
ATACGCGARGAACCTTACC
113-967F-AQ:
(SEQ ID NO: 3)
CTAACCGANGAACCTYACC
114-967F-S:
(SEQ ID NO: 4)
CAACGCGMARAACCTTACC
115-1046R-S:
(SEQ ID NO: 5)
CGACRRCCATGCANCACCT

Absolute numbers of bacteria in the samples were then approximated as DNA amount in a sample/DNA molecule mass of bacteria.

Immunofluorescence: For fluorescent visualization of bacteria, colon samples were fixed using freshly prepared Carnoy's fixative (60% EtOH, 30% Chloroform, 10% glacial acetic acid) for 2 hours, at 4° C., followed by a wash with 100% EtOH and storage in 100% EtOH until paraffin embedding and section. Colon sections were hybridized with 16S probe at a concentration of 10 ng/μl over night at 50° C., diluted in hybridization buffer (20 mM Tris-HCl (PH 7.4), 0.9M NaCl, 0.1% SDS.

Probe sequence: GCTGCCTCCCGTAGGAGT ((SEQ ID NO: 6), dual labeled, 5′ CAL flour Red 610, 3′ BHQ-2.

For co-immunostaining, tissue sections were blocked at room temperature for 30 min in PBS^{-/-}, 0.05% Triton and 20% normal horse serum, followed by incubation with anti-Muc2 antibody (H300 Santa Cruz), 1:200 in PBS^{-/-}, 0.05% Triton and 2% normal horse serum, over night at 4° C. Sections were then washed in PBS and incubated with a secondary Ab, 1:400, 2 hrs at room temperature. Samples were then washed and mounted. Visualization was performed using a Nikon Eclipse Ti microscope.

Taxonomic microbiota analysis: Frozen fecal samples were processed for DNA isolation using the MoBio PowerSoil kit according to the manufacturer's instructions. For the 16S rRNA gene PCR amplification, ing of the purified fecal DNA was used for PCR amplification. Amplicons spanning the variable region 1/2 (V1/2) of the 16S rRNA gene were generated by using the following barcoded primers: Fwd 5′-XXXXXXXXAGAGTTTGATCCTGGCTCAG-3′ (SEQ ID NO: 7), Rev 5′-TGCTGCCTCCCGTAGGAGT-3′ (SEQ ID NO: 8), where X represents a barcode base. The reactions were subsequently pooled and cleaned (PCR clean kit, Promega), and the PCR products were then sequenced on an Illumina MiSeq in 500 bp paired-end method. The reads were then processed using the QIIME (Quantitative Insights Into Microbial Ecology, www(dot)qiime(dot)org) analysis pipeline as described (Levy et al., 2015). In brief, fasta quality files and a mapping file indicating the barcode sequence corresponding to each sample were used as inputs, reads were split by samples according to the barcode, taxonomical classification was performed using the RDP-classifier, and an OTU table was created. Closed-reference OTU mapping was employed using the Greengenes database.

Rarefaction was used to exclude samples with insufficient count of reads per sample. Sequences sharing 97% nucleotide sequence identity in the V2 region were binned into operational taxonomic units (97% ID OTUs). For beta-diversity, weighted unifrac measurements were plotted according to the first two principal coordinates based on 10,000 reads per sample.

Metagenomic analysis: Metagenomic reads containing Illumina adapters were filtered, low quality reads were filtered and low quality read edges were trimmed. Host DNA was detected by mapping with GEM (Marco-Sola et al., 2012) to the mouse genome with inclusive parameters, and those reads were removed. The present inventors assigned length-normalized RA of genes, obtained by similar mapping with GEM to a reference catalog (Li et al., 2014), to KEGG Orthology (KO) entries (Kanehisa and Goto, 2000), and these were then normalized to a sum of 1. They calculated RA of KEGG modules and pathways by summation. Only samples with >100K metagenomics reads were considered.

Quality control of metagenomic reads and removal of host DNA: Trimmomatic (Bolger et al., 2014) was applied with the following parameters: ILLUMINACLIP:<Trueseq3 adapters fasta file>:2:30:10 LEADING:25 TRAILING:25 MINLEN:50. Host DNA was removed by mapping to the mouse genome (mm10, downloaded from genome(dot)ucsc(dot)edu) and removing any mapped reads (see section below).

Functional assignment of metagenomic reads: Reads mapped to the gut microbial gene catalog were assigned a KEGG (Kanehisa and Goto, 2000). Genes were subsequently mapped to KEGG modules and pathways. For the KEGG pathway analysis, only pathways whose gene coverage was above 0.2 were included. Bacterial assignment to metabolic pathways was done by mapping of metagenomic reads to genes from the respective metagenomic modules. Mapped reads were extracted and re-mapped to a bacterial genomes database. Reads that were successfully mapped were grouped into genera, and those not mapped were marked as ‘unknowns’.

RNA-seq processing and analysis: A derivation of MARS-seq was used as described (Jaitin et al., 2014), developed for single-cell RNA-seq to produce expression libraries with a minimum of two replicates per group. The RNA-seq reads were aligned to the mouse reference genome (NCBI 37, mm9) using TopHat v2.0.13 with default parameters (Trapnell et al., 2009). Duplicate reads were filtered if they aligned to the same base and had identical UMIs. Expression levels were calculated and normalized for each sample to the total number of reads using HOMER software (www(dot)homerdotsalk(dot)edu) with the command “analyzeRepeats.pl ma mm9 -d [sample files]-count 3utr -condenseGenes” (Heinz et al., 2010). KEGG analysis was done using DAVID (Dennis et al., 2003). For the generation of heatmaps, genes were normalized to their mean expression across all time points.

iChIP-IVT: A recently developed protocol for ChIP-seq involving amplification by in-vitro transcription was used (Gury-BenAri et al., 2016). Sorted epithelial cells were cross-linked for 8 min in 1% formaldehyde and quenched for 5 min in 0.125 M glycine prior to sorting. Sorted and frozen cell pellets were lysed in 0.5% SDS and sheared with the NGS Bioruptor Sonicator (Diagenode). Sheared chromatin was immobilized on 12 μl Dynabeads Protein G (Invitrogen) with 1.3 μg of anti-H3 antibody (ab1791). Magnetized chromatin was then washed with 10 mM Tris-HCl supplemented with 1× protease inhibitors. Chromatin was end repaired, dA-tailed and ligated with 5 μl of 0.75 μM partial Illumina Read2 sequencing adapters containing T7 polymerase promotor. Indexed chromatin was pooled, split to 3 IP pools and incubated with 2.5 μg anti-H3K4me2 antibody (ab32356)/anti-H3K4me3 (Millipore, 07-473)/anti-H3K27Ac (ab4729) at 4° C. for 3h and for an additional hour with Protein G magnetic beads (Invitrogen). Magnetized chromatin was washed and reverse cross-linked. DNA was subsequently purified with 1.65× SPRI. In vitro transcription step of linear amplification were introduced to produce RNA transcripts out of the DNA fragments using the T7 High Yield RNA polymerase IVT kit (NEB). After IVT, DNAse treatment was performed to eliminate the DNA fragments. Next, a partial Illumina Readl sequencing adapter that includes a pool barcode was single strand ligated to the fragmented RNA using a T4 RNA ligase I (New England Biolabs). The ligated product was reverse transcribed using Affinity Script RT enzyme (Agilent) and a primer complementary to the ligated adapter. The library was completed and amplified through a PCR reaction with 0.5 μM of each primer and PCR ready mix (Kapa Biosystems). The forward primer contains the Illumina P5-Readl sequences and the reverse primer contains the P7-Read2 sequences. DNA concentration was measured with a Qubit fluorimeter (Invitrogen) and mean molecule size was determined with 2200 TapeStation analyzer (Agilent) and library quality was further determined by qPCR. Primer sequences are identified in Table 1, herein below:

TABLE 1

| Primer name | Sequence and modifications |
|---|---|
| Chromatin | CGATTGAGGCCGGTAATACGACTCACTATAGGGGCGACGTG |
| first indexing | TGCTCTTCCGATCTXXXXXXXXT (SEQ ID NO: 9) modified with |
| adapter | a C3 spacer (blocker) at the 5′. XXXXXXXX is the barcode for |
| | sample multiplexing. |
| Ligation | XXXXNNNNNAGATCGGAAGAGCGTCGTGTAG (SEQ ID NO: |
| adapter | 10) |
| | modified with a phosphate group at 5′ and a C3 spacer (blocker) at the |
| Second primer | TCTAGCCTTCTCGCAGCACATC (SEQ ID NO: 11) |
| P5_Rd1 | AATGATACGGCGACCACCGAGATCTACACTCTTTCCCTACA |
| | C (SEQ ID NO: 12) |
| P7_Rd2 | CAAGCAGAAGACGGCATACGAGATGTGACTGGAGTTCAGA |
| | C (SEQ ID NO: 13) |

Processing of ChIP-seq: Reads were aligned to the mouse reference genome (mm9, NCBI 37) using Bowtie2 aligner version 2.2.5 (Langmead et al., 2009) with default parameters. The Picard tool MarkDuplicates from the Broad Institute (broadinstitute(dot)github(dot)io/picard/) was used to remove PCR duplicates. To identify regions of enrichment (peaks) from ChIP-seq (H3K4me2, H3K4me3, H3K27ac), we used the HOMER package makeTagDirectory followed by findPeaks command with the histone parameter or 500 bp centered regions, respectively (Heinz et al., 2010). Union peaks file were generated for each of H3K4me2 and H3K4me3 by combining and merging overlapping peaks in all samples.

Chromatin analysis: The read density (number of reads in 10 million total reads per 1000 bp) was calculated in each region from the union peaks files. The region intensity was calculated by quantile normalization (across samples) of the read density in log base 2 (log2(x+1)). The present inventors considered promoters to be H3K4me3 regions within +/−2000 bp of a TSS and candidate enhancers to be distal H3K4me2 regions. Enhancers were assigned to the nearest gene within 50 kb. The activity level of promoters and enhancers was determined by the H3K27ac intensity within the H3K4me3 or H3K4me2 region, respectively.

Gene tracks and normalization: All gene tracks were visualized as bigWig files of the combined replicates normalized to 10,000,000 reads and created by the HOMER algorithm makeUCSCfile (Heinz et al., 2010). For visualization, the tracks were smoothed by averaging over a sliding window of 500 bases.

Metabolomics: Fecal samples were collected, immediately frozen in liquid nitrogen and stored at −80° C. Samples were prepared using the automated MicroLab STAR® system from (Hamilton). To remove protein, dissociate small molecules bound to protein or trapped in the precipitated protein matrix, and to recover chemically diverse metabolites, proteins were precipitated with methanol. The resulting extract was divided into five fractions: one for analysis by UPLC-MS/MS with positive ion mode electrospray ionization, one for analysis by UPLC-MS/MS with negative ion mode electrospray ionization, one for LC polar platform, one for analysis by GC-MS, and one sample was reserved for backup. Samples were placed briefly on a TurboVap® (Zymark) to remove the organic solvent. For LC, the samples were stored overnight under nitrogen before preparation for analysis. For GC, each sample was dried under vacuum overnight before preparation for analysis.

Data Extraction and Compound Identification: Raw data was extracted, peak-identified and QC processed using Metabolon's hardware and software. Compounds were identified by comparison to library entries of purified standards or recurrent unknown entities. Metabolite

Quantification and Data Normalization: Peaks were quantified using area-under-the-curve. For studies spanning multiple days, a data normalization step was performed to correct variation resulting from instrument inter-day tuning differences.

For targeted analysis of polyamines and amino acids, 50 μl of serum were diluted 100 μl 0.1% formic acid in acetonitrile. After cooling on ice for 10 minutes, the samples were centrifuged, and 10 μl of the supernatant was added to 70 μl of borate buffer and derivatized using the AccQTag method (Boughton et al., 2011). The LC-MS/MS instrument consisted of Acquity I-class UPLC system and Xevo TQ-S triple quadrupole mass spectrometer (Waters). Chromatographic separation and mass detection were carried out in the conditions recently described (Zwighaft et al., 2015) with adjustments to obtain the highest signal for each compound.

Histology: Sections from the left lobe of the liver were fixed in paraformaldehyde and embedded in paraffin for staining with H&E. Subsequently, sections were examined by a blinded veterinary pathologist and scored for necrosis and hemorrhage on a scale from 0 (healthy) to 3 (most severe).

Statistical analysis: Data are expressed as mean ±SEM. Cycling behavior was assessed using JTK_cycle (Hughes et al., 2010), with oscillations tested for a 24 hour period length. Unless stated otherwise, elements with p<0.05 and q<0.1 were considered significant. For metabolites, q<0.2 was considered significant. For two-group comparisons, Mann-Whitney U-test was used and comparisons with p<0.05 were considered significant. Analysis of shared and unique oscillatory transcripts was performed on balanced groups to account for the sensitivity of JTK_cycle to the number of replicates. This was done by random sub-sampling of samples at each time point, performed 250 times. Hence, average numbers are presented. Significance and numbers of the fraction of shared oscillating transcripts out of total oscillating transcripts in colon and liver tissues was determined by permutations followed by re-running of the JTK_cycle algorithm. In each permutation samples were randomly selected at each time point from both antibiotic-treated and controlled mice, maintaining balance both in the number of replicates and in the number of replicates belonging to each group across time points.

Results

The Biogeography of the Intestinal Microbiota Undergoes Diurnal Oscillations

Since the commensal bacteria most strongly affecting the host are located in proximity to the intestinal mucosal surface, the present inventors studied the biogeographical aspects of microbiome diurnal rhythmicity, by analyzing fluctuations in the abundance, composition, and function of epithelial-adherent commensal bacteria in the colon over the course of two days (FIG. 1A). All mice were fed ad libitum and housed under strict 24-hour dark-light conditions, with lights being kept on for 12 hours (Zeitgeber times (ZT) 0-12). Scanning electron microscopy (SEM) imaging of proximal colons revealed daily fluctuations in the amount of commensals in tight association with the intestinal epithelium (FIGS. 1B, 8A, and 8B, p<10⁻⁵, JTK_cycle). To determine the absolute numbers of mucosal-associated bacteria, the present inventors extensively cleared proximal colons from luminal content to retain only the mucosal niche, and confirmed the successful isolation of epithelial-proximal bacteria by detection of mucus-resident commensals, including Mucispirillum schaedleri (Robertson et al., 2005). In line with the observations made by SEM, the numbers of bacteria colonizing the epithelial niche, as quantified by qPCR of the total 16s rDNA pool, underwent marked diurnal changes, with epithelial layer adherence in the dark phase being up to 10-fold higher than in the light phase (FIG. 1C, p<10⁻⁶). These results were confirmed by using a sampling frequency of 4 hours (FIG. 8C). To assess whether the bacterial composition in the mucosal niche likewise underwent temporal fluctuations, 16S rDNA sequencing of epithelial-associated communities harvested at different times of the day was performed.

Indeed, the global bacterial composition featured marked diurnal oscillations (FIGS. 1D and 1E), such that the bacterial community localized to the intestinal mucosa at any time point was more similar to the one present 24 hours earlier than to any other time point in between (FIGS. 8D-8F). To determine the absolute numbers of bacteria colonizing the mucosal niche 16S sequencing was then combined with qPCR quantification (FIG. 8G). 148 out of 633 detected operational taxonomic units (OTUs) featured rhythmic patterns of epithelial adherence (FIG. 1F, p<0.05, q<0.1), including Mucispirillum schaedleri (FIG. 1G), Lactobacillus reuteri (FIG. 8H), and Bacteroides acidifaciens (FIG. 8I). Together, these results suggest that the host mucosa is exposed to diurnally fluctuating numbers and species of bacteria over the course of a day.

Host and Microbial Factors Regulate Bacterial Mucosal-Associated Oscillations

To determine the mechanisms driving rhythmic bacterial mucosal localization, metagenomic sequencing of the mucosal microbial community was performed every 6 hours over the course of 48 hours. First rhythmic changes in the abundance of bacterial KEGG genes in epithelial proximity were assessed. 404 out of 1552 genes significantly oscillated in their relative abundances, among them members of the flagellar gene operons (FIGS. 1H and 1I, p<0.05, q<0.1). The present inventors furthermore assigned KEGG modules and pathways to the microbial genes (FIGS. 2A and 9A). Interestingly pathways involved in mucus degradation (FIGS. 1H, 2A, and 9A) and bacterial motility (FIGS. 1H, 2A, and 9A) were found to be among the microbial functions most significantly oscillating in relative abundance, as exemplified by bacterial chemotaxis (FIG. 2B) and flagellar assembly (FIG. 9B). To determine the bacterial species driving pathway-level rhythmicity in the mucosal community, those OTUs that contributed to oscillating genes within the bacterial chemotaxis and flagellar assembly pathways were examined. Notably, the majority of such species belonged to Deferribacteraceae, a bacterial family which itself featured robust oscillations in mucosal abundance (FIG. 9C).

The present inventors hypothesized that rhythmic bacterial movement and mucus invasion might contribute to the daily fluctuations in commensal inhabitation of the mucosal niche. They therefore performed a time course of microbiota imaging by 16S in situ hybridization every 6 hours over the course of two days, using a mucus-preserving fixation method and co-staining for Muc2 protein in order to visualize mucus production. Expectedly, they found a two-layered mucus structure, the inner of which is largely free of bacterial colonization (FIG. 9D). Interestingly, the thickness of the mucus layer, as well as the degree of microbial penetration into the mucus layer and thus the width of spatial separation between the host intestinal epithelium and the commensal bacteria, underwent rhythmic fluctuations (FIGS. 2C, 2D, and 9D). Microbial proximity to the mucosal surface was highest during the dark phase (FIG. 2C, p<10⁻²⁰), corroborating the results obtained by electron microscopy and 16S qPCR.

The spatial segregation between epithelial layer and commensal bacteria is maintained by the production of mucus and antimicrobial peptides. Recently, it was found that mice lacking RegIIIγ feature enhanced bacterial colonization in direct proximity to the intestinal epithelium (Loonen et al., 2014; Vaishnava et al., 2011). Indeed, the present inventors found an impairment of spatial separation between the epithelial layer and the microbiota and abrogated diurnal rhythmicity in the number of mucosal-resident bacteria in RegIIIγ-deficient mice, as determined by in-situ hybridization (FIGS. 2E and 9E), 16S qPCR (FIG. 2F), and 16S sequencing (FIG. 9F).

To determine the contribution of the host circadian machinery to the circadian bacterial adherence patterns, Per1/2^{-/-} mice that are devoid of the core molecular clock we utilized (Adamovich et al., 2014). A marked loss of bacterial adherence oscillations in Per1/2^{-/-} mice was noted, (FIG. 2G), indicating that host circadian rhythms are indispensable for the maintenance of microbiota biogeographical rhythms. To disentangle the dysfunctional molecular clock from abrogated feeding rhythms noted in these mice (Neufeld-Cohen et al., 2016), timed-feeding experiments were performed, in which Per1/2^{-/-} mice had access to food only during the light phase. Notably, this treatment restored microbiota oscillations, both in the amount and composition of the mucosal-resident bacterial community (FIGS. 2H, 2I, 9G, and 9H). These results identify feeding time as a major driver of microbial biogeography. In support of this notion, wild-type mice that were fed either only during the dark or only during the light period showed phase-reversed microbial attachment rhythms (FIG. 9I).

Together, these results identified several host and microbial factors jointly contributing to diurnal variations in epithelial proximity of commensal bacteria, including the host circadian clock through regulation of feeding rhythms, RegIlly-mediated barrier function, as well as rhythmic bacterial motility and mucus degradation.

Microbiota Ablation Reprograms the Intestinal Circadian Transcriptome

The impact of the diurnally fluctuating microbiome on the intestinal epithelium was next assessed. To this end, the bacterial ecosystem was disrupted by administering broad-spectrum antibiotics and the host circadian transcriptional and epigenetic intestinal program was assessed (FIG. 3A). Expectedly, antibiotic treatment abrogated both the number of mucosal-associated bacteria and their oscillatory behavior, as determined by 16S qPCR and scanning electron microscopy(FIGS. 3B, 3C, and 10A). Furthermore, the remaining antibiotic-persistent epithelial-proximal microbiota lost its diurnal rhythms in composition (FIGS. 3D, 3E, 10B, and 10C).

To determine the impact of microbiota disruption on the rhythmic transcriptome of the host, comparative RNA-sequencing was performed of colonic tissue from control and antibiotics-treated mice every 6 hours over the course of two light-dark cycles (FIG. 3A). First, the detection of known effects of microbiota depletion on colonic transcription by qPCR was confirmed (FIG. 10D). Next, host transcript rhythmicity was evaluated on a global level. Interestingly, while robust circadian oscillations were detected in several hundred genes in both control and antibiotics-treated groups, the identity of the most significantly oscillating genes was markedly different between the groups, which was observed with both 6 hours and 4 hours sampling frequency (FIGS. 3F-3I and 10E). Behavioral rhythms of the host, including feeding rhythmicity, persisted throughout the antibiotic treatment (FIGS. 10F, 10G, and data not shown), ruling out loss of rhythmic food intake as the reason for the observed transcriptional reprogramming. Loss and gain of oscillatory host transcripts was unrelated to expression levels, since average expression of the affected genes was not influenced by antibiotic treatment (FIG. 10H), suggesting that transcript oscillation was an independently regulated feature.

To determine the functionality of lost, gained, and shared transcript oscillations, KEGG pathways were assigned to each group of genes. The pathway most significantly enriched among the oscillatory transcripts shared between antibiotic treated and control mice was the core circadian clock (p<10⁻⁹), indicating that the function of the host peripheral clock machinery was not intrinsically dependent on the presence of an intact microbiota (FIGS. 3G and 3J). Transcripts that lost their oscillations in the absence of the microbiota mainly belonged to nucleotide metabolism and cell cycle pathways (FIGS. 3H and 3K). Most remarkable and unexpected, however, were the functionalities that gained rhythmicity upon microbiota depletion, which included major metabolic pathways like pyruvate metabolism, glutathione metabolism, and the TCA cycle (FIGS. 3I and 3L). Interestingly, similar pathways were significantly oscillating in the mucosal microbiome under homeostatic conditions (FIG. 10I), potentially suggesting that upon microbiota depletion, the host may acquire compensatory oscillatory programs in functionalities that are normally performed by the microbiota in a rhythmic manner. These data indicate that a large set of rhythmic transcripts in the colon is influenced by the intestinal microbiota, and that microbiota depletion incites de novo oscillatory programs in the host.

Microbiota Ablation Reprograms Circadian Chromatin Dynamics

To gain insight into the mechanisms by which the microbiota influences the programming of colonic transcriptome oscillations, the gene regulatory mechanisms underlying rhythmic transcription in intestinal epithelial cells were investigated. Clock-driven transcriptome oscillations in the liver are accompanied by rhythmic genome-wide remodeling of the chromatin state (Koike et al., 2012; Vollmers et al., 2012). The present inventors therefore sought to characterize the circadian epigenetic landscape of intestinal epithelial cells, and to determine whether the microbiota influences rhythmic chromatin remodeling in a genome-wide manner. To this end, they performed a time-course of chromatin immunoprecipitation followed by next-generation sequencing (ChIP-seq) profiling on purified intestinal epithelial cells every 6 hours over two days (FIG. 4A). They assayed several histone modifications including trimethylation of histone H3 at lysine 4 (H3K4me3), H3K4me2, and H3K27 acetylation (H3K27ac) to determine the global landscape of enhancers (distal regions marked by H3K4me2), promoters (characterized by the enrichment of H3K4me3) and active transcription, indicated by H3K27Ac marks (FIG. 4A).

Given the close association of circadian promoters and rhythmic transcription (Koike et al., 2012; Vollmers et al., 2012), the present inventors first examined the genome-wide architecture of active promoters. To this end, they identified H3K27ac peaks in H3K4me3 regions and assessed rhythmicity over the course of two days by JTK_cycle. Significant oscillations in 525 active promoters (FIG. 11A, p<0.05), including the loci of the canonical clock genes (FIG. 11B) were found. False-positive detection of stochastic oscillations was ruled out by comparing rhythmic histone marks to the genomic background of whole-cell extracts (FIG. 11A). Next, using this map of genome-wide oscillations in active promoters, the impact of the microbiome on the temporal organization of the chromatin landscape was assessed by performing a ChIP-seq time-course on antibiotics-treated mice (FIG. 4A). Remarkably, and in agreement with the gene expression data, cycling behavior of promoter marks in both control and antibiotics groups were found, but the identity of the oscillating loci was largely distinct (FIGS. 4B and 4C), with 491 promoters losing rhythmicity upon microbiota depletion, but 477 loci developing de novo rhythmic behavior (FIG. 4C). Among the genes with shared promoter rhythmicity were genes associated with the core clock, as exemplified by Dbp (FIGS. 4D and 11C), while the loss and gain of oscillations affected genes across various functional groups (FIGS. 4E and 4F). The examples of Nrldl (shared oscillation), Mxdl (lost oscillation), and Cxadr (de novo oscillation) illustrate the association of loss and gain of H3K27ac rhythmicity at promoter regions with transcript oscillations (FIG. 11D).

In addition to active promoters, circadian transcription is closely associated with cyclic enhancer regions (Koike et al., 2012; Vollmers et al., 2012). The present inventors therefore also focused on rhythmic H3K4me2 peaks and investigated the impact of the microbiota on the diurnal enhancer landscape. As with circadian promoters, overall rhythmicity was not affected by microbiota depletion (FIGS. 4G and 11E). However, the loci of both rhythmic enhancer usage (H3K4me2) and enhancer activity (H3K4me2 +H3K27ac) substantially differed between both scenarios, with several hundreds of enhancers losing and gaining rhythmicity upon antibiotic treatment (FIGS. 4H and 11F). When active enhancers (H3K27ac oscillations at intergenic and intragenic H3K4me2 loci) were examined, a strong association of cycling genes with cycling enhancer marks was observed in the antibiotics setting, suggesting that the rhythmic behavior of such enhancers becomes active upon changes in the state of microbial colonization. Such enhancer activity-driven de novo oscillation was observed, for example, in the calcium-binding protein S100a10 (FIGS. 4I and 4J), while loss of rhythmicity is exemplified by the phosphatase Ctdsp2 (FIGS. 4K and 4L). Malate dehydrogenase (Mdhl) represented a further example in which enhancer activity (H3K4me2 +H3K27ac), rather than enhancer usage (H3K4me2 alone), regulated the activation of oscillating transcription upon microbiota depletion (FIGS. 11G and 11H). Together, these data demonstrate an impact of the microbiota on the architecture of oscillating chromatin modifications and suggest that microbial colonization influences rhythmic promoter and enhancer activity to drive rhythmic gene expression.

Microbial Attachment Influences the Intestinal Circadian Transcriptome

The above antibiotic treatment experiments suggested that the microbiome regulates oscillations in the host transcriptome and epigenome, but could not distinguish between the involvement of bacterial attachment to the mucosa or its mere presence in the intestine. To differentiate between these two possibilities, germ-free mice were mono-colonized with adherent and non-adherent variants of the same bacterial species (FIG. 5A). Segmented filamentous bacteria (SFB) indigenous to mice (mSFB) or rats (rSFB) were used, previously shown to feature differential adherence to the intestinal epithelium (Atarashi et al., 2015). Indeed, mSFB was found in more than 10-fold higher numbers in the mucosal-proximal layer of mice as compared to rSFB (FIG. 5B). Despite these differences in mucosal colonization, both types of SFB featured phase-shifted rhythmic patterns of absolute numbers in epithelial proximity over the course of 48 hours (FIG. 5C).

This system enabled the present inventors to determine the impact of differential rhythmic bacterial attachment of the colonic circadian transcriptome in mice mono-colonized with mSFB (featuring a rhythmic mucosal abundance), mice mono-colonized with rSFB (featuring a rhythmic yet markedly reduced mucosal abundance), and mice lacking RegIlly (featuring an abundant but non-rhythmic mucosal microbiome, FIG. 2F). The identity of oscillating genes in these mice were comparedwith those featured in either antibiotics-treated or control mice (FIG. 3F). Notably, the oscillating transcripts of mSFB-mono-colonized mice were most similar to wild-type controls (FIG. 5D) while those of Reg3g^{-/-} mice showed the strongest overlap with those of antibiotics-treated animals, suggesting that rhythmic bacterial adherence per se was involved in determining the program of rhythmically expressed genes. Mono-colonization with rSFB resulted in an intermediate oscillatory program, possibly reflecting its combination of reduced adherence yet oscillating microbiome activity (FIG. 5D). These alterations in rhythmic transcription did not influence oscillations of the core molecular clock, as exemplified by Dbp expression (FIG. 5E). Apart from the members of the circadian clock, mSFB mono-colonization was associated with rhythmic transcription of genes belonging to DNA replication, cell cycle, and nucleotide turnover pathways (FIG. 5F), similar to control mice harboring a conventional microbiome (FIG. 3K). Together, these results suggest that rhythmic bacterial adherence drives a program of transcriptional oscillations that is altered upon interference with the rhythmic biogeography of the microbiota.

Microbiota Ablation Reprograms the Hepatic Circadian Transcriptome

The present inventors next examined whether the impact of the microbiota on oscillatory programs of the host reaches beyond the gastrointestinal tract. To this end, they performed RNA-seq analysis of livers from antibiotics-treated or control mice taken at 6 hour intervals over two days and assessed for rhythmicity using JTK_cycle (FIG. 5G). Similar to what had been observed in the colon, antibiotics-mediated microbiota disruption reprogrammed liver transcriptome oscillations (FIGS. 5H and 12A-C), without changing the mean expression levels of the affected genes (FIG. 12D). As in the gut, the canonical clock components maintained their rhythms (FIGS. 51, 5J, 12A, and 12E), as did genes involved in hepatic drug metabolism, insulin signaling, and multiple other metabolic functions (FIGS. 5I and 12A). In total, 375 out of 1306 hepatic transcripts cycled only in the control group (p<0.05, q<0.1; FIG. 5H), including genes involved in oxidative phosphorylation and other catabolic pathways (FIGS. 5K and 12B). For instance, the gene encoding glucose phosphate isomerase-1 (Gpil) lost detectable rhythmicity after antibiotic treatment (FIG. 5K). In contrast, 912 transcripts developed de novo rhythmicity (p<0.05, q<0.1; FIGS. 5H and 12C), many of which were involved in amino acid metabolism and fatty acid metabolism, including elements of the PPARγ signaling pathway (FIG. 5L) (Murakami et al., 2016). To corroborate that this transcriptional reprogramming was due to microbiota depletion, rather than direct effects of the antibiotics on the liver, the profile of circadian hepatic transcriptome of germ-free mice was compared to the profile found in colonized control mice and mice treated with broad-spectrum antibiotics. A high concordance of oscillating transcripts was found in livers from germ-free mice with those from antibiotics-treated mice, while the overlap with non-treated control mice was smaller (FIG. 12F). Taken together, these results highlight the microbiota as being critical for maintaining the homeostatic rhythmic transcription in the liver.

The Microbiota Programs the Hepatic Transcriptome Through Systemic Metabolome Oscillations

The present inventors next sought to determine the mechanisms by which the gut microbiota distally orchestrates hepatic transcriptome oscillations. Metabolism has emerged as a major regulator of the epigenetic control of gene expression, and several prominent examples of close interaction between metabolites and the circadian clock have been unraveled (Asher and Sassone-Corsi, 2015). To gain insight into metabolites as potential mediators of the effect that the microbiota exerts on rhythmic gene expression, the present inventors first determined the temporal dynamics of the intestinal metabolome by metabolomic profiling in wild-type mice every 6 hours over the course of two light-dark cycles (FIG. 5G). They detected significant oscillations across diverse chemical groups, including lipids, amino acids, carbohydrates, vitamins, nucleotides, and xenobiotics (FIG. 6A), as exemplified by the carbohydrate xylose, the dipeptide valylglutamate, and the histidine derivative ergothioneine (FIGS. 13A-C). Oscillatory behavior was detected along biosynthetic pathways, such as the conversion between the polyamine ornithine and the amino acid proline (FIG. 6B, p<10⁻³). In the case of biotin, a bacterial-derived vitamin with essential functions for host physiology, rhythmicity along the biosynthetic pathway was noted (FIG. 6C, p<10^{Δ5}), involving the conversion of 7,8-diaminononanoate to dethiobiotin (catalyzed by bioD), and the subsequent production of biotin (catalyzed by bioB). Furthermore, rhythmic elements characterized the glycolytic conversion of sucrose to lactate (FIG. 13D) were noted. These results demonstrate that multiple microbiota-derived and -modulated metabolites undergo diurnal rhythms, suggesting a potential mechanism by which microbiota metabolism may influence the diurnal transcriptional landscape of the host.

To determine interactions between oscillating intestinal metabolites and systemic host circadian activity, the temporal behavior of serum metabolites were profiled over a 48-hour time course (FIG. 5G). Amino acids and polyamines were focused on, given their prominent rhythmicity in the lumen of the gastrointestinal tract and their known regulatory involvement in the activity of the circadian clock (Zwighaft et al., 2015). Notably, a high degree of concordance was detected between rhythmicity of a particular metabolite in the serum and in the intestinal lumen (R²=0.415, p<10⁻³; FIG. 6D). Some metabolites featured phase-shifted oscillations in the serum, as demonstrated for threonine, ornithine, proline, and a-aminobutyric acid (FIGS. 6E and 13E-13G). To test whether the microbiota was involved in the orchestration of this serum rhythmicity of amino acids and polyamines, the present inventors profiled their diurnal pattern in antibiotics-treated and germ-free mice. Remarkably, absence of the microbiota abolished rhythmicity in any of the examined metabolites (FIG. 6F). For instance, antibiotics-treated and germ-free mice did not show diurnal rhythms in the serum levels of ornithine (FIGS. 6G and 6H). Furthermore, while arrhythmic Per1/2^{-/-} mice did not feature any detectable serum amino acid or polyamine oscillations, restoring microbiota oscillations in these mice by timed feeding reinstated metabolite rhythms to the level of wild-type controls (FIGS. 6F and 13H). This was seen, for instance, in ornithine and the biosynthetically related metabolites arginine and proline (FIGS. 6I, 13I, and 13J).

To further investigate whether the levels and fluctuations of intestinal amino acids and polyamines were causally involved in the reprogramming of hepatic transcript oscillations, a dietary intervention was performed in which mice were fed a polyamine-deficient (PD) diet for 4 weeks, followed by serum metabolite analysis and RNA-seq of hepatic tissue every 6 hours over two days (FIG. 7A). Indeed, PD diet abrogated homeostatic circadian oscillations in serum amino acids and polyamines (FIGS. 7B and 14A-14C). Remarkably, this was accompanied by reprogramming of the hepatic circadian transcriptome, including the loss and de-novo gain of several hundreds of rhythmic genes (FIGS. 7C-7G). The genes oscillating in mice fed a PD diet showed the highest concordance with those measured in antibiotics-treated mice, and to a much lesser extent to non-treated control mice (FIG. 7D). Together, these results suggest that diet and the gut microbiota are central contributors to the maintenance of systemic metabolome rhythms, thereby impacting the programming of the circadian hepatic transcriptome. In demonstrating these links between diet, microbiome, circulating metabolites, and the liver, the present inventors focused as proof-of-concept on amino acids and polyamines, while similar impacts by other metabolite families merit further studies.

Microbiota-Mediated Reprogramming of the Circadian Transcriptome Alters Diurnal Hepatic Detoxification

Finally, the consequences of microbiota-mediated reprogramming of the daily sequence of gene expression for the physiological function of the liver was considered. The time of day is known to greatly affect hepatic drug metabolism, including the detoxification of acetaminophen (acetyl-para-aminophenol, APAP) (Kim and Lee, 1998). Additionally, the microbiota has been implicated in APAP metabolism (Clayton et al., 2009). The present inventors therefore administered APAP at different circadian times (ZTO versus ZT12) and assessed APAP-induced hepatotoxicity by the measurement of liver enzyme release as well as liver histology. In line with previous reports (Johnson et al., 2014; Kim and Lee, 1998), mice featured dramatically exacerbated liver toxicity when APAP was injected at ZT12 as compared to ZTO (FIG. 7H-7K). This diurnal variation was clock-dependent, as Per1/2^{-/-} mice did not feature differential hepatotoxicity between ZT 0 and ZT12 (FIGS. 14D-14G). Remarkably, antibiotics-treated or germ-free mice lost this diurnal variation in the severity of APAP-induced hepatotoxicity, and featured low and comparable levels of liver damage at different times of the day (FIG. 7H-7K), manifesting as a significantly lower aminotransferase activity, reduced liver necrosis, and improved histopathological score. Together, these results suggest that homeostatic microbiota rhythms and microbiota-mediated maintenance of the circadian transcriptome is necessary to maintain normal diurnal activity in hepatic drug metabolism.

The present data provides insights into the functional consequences of the microbiome-mediated disruption of normal circadian physiology. The data show that antibiotic treatment or disruption of circadian feeding behavior leads to a multi-faceted disruption of microbiota diurnal rhythmicity, thereby generating a temporal de-synchronization of circadian liver functions. The metabolism of APAP by both host and microbiota is one such example of a diurnally shifting housekeeping activity, in which the time of exposure to an APAP overdose determines the production level of hepatotoxic APAP degradation products. This diurnal activity, and its functional consequences during APAP intoxication, is abrogated in antibiotic-treated or germ-free mice, highlighting the microbiome as major contributor to the chronopharmacology of drugs, environmental xenobiotics, and dietary components. Together, understanding of the role of the microbiome in the diurnal adaptation of peripheral organ activity may prove instrumental for understanding and treating human conditions associated with disruption of the microbiota and the circadian clock.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

Adamovich, Y., Rousso-Noori, L., Zwighaft, Z., Neufeld-Cohen, A., Golik, M., Kraut-Cohen, J., Wang, M., Han, X., and Asher, G. (2014). Circadian clocks and feeding time regulate the oscillations and levels of hepatic triglycerides. Cell metabolism 19, 319-330.

Aguilar-Arnal, L., Hakim, O., Patel, V.R., Baldi, P., Hager, G. L., and Sassone-Corsi, P. (2013). Cycles in spatial and temporal chromosomal organization driven by the circadian clock. Nature structural & molecular biology 20, 1206-1213.

Asher, G., Gatfield, D., Stratmann, M., Reinke, H., Dibner, C., Kreppel, F., Mostoslavsky, R., Alt, F. W., and Schibler, U. (2008). SIRT1 regulates circadian clock gene expression through PER2 deacetylation. Cell 134, 317-328.

Asher, G., and Sassone-Corsi, P. (2015). Time for food: the intimate interplay between nutrition, metabolism, and the circadian clock. Cell 161, 84-92.

Atarashi, K., Tanoue, T., Ando, M., Kamada, N., Nagano, Y., Narushima, S., Suda, W., Imaoka, A., Setoyama, H., Nagamori, T., et al. (2015). Th17 Cell Induction by Adhesion of Microbes to Intestinal Epithelial Cells. Cell 163, 367-380.

Bolger, A. M., Lohse, M., and Usadel, B. (2014). Trimmomatic: a flexible trimmer for Illumina sequence data. Bioinformatics 30, 2114-2120.

Boughton, B. A., Callahan, D. L., Silva, C., Bowne, J., Nahid, A., Rupasinghe, T., Tull, D. L., McConville, M. J., Bacic, A., and Roessner, U. (2011). Comprehensive profiling and quantitation of amine group containing metabolites. Anal Chem 83, 7523-7530.

Caporaso, J. G., Kuczynski, J., Stombaugh, J., Bittinger, K., Bushman, F. D., Costello, E.K., Fierer, N., Pena, A. G., Goodrich, J. K., Gordon, J. I., et al. (2010). QIIME allows analysis of high-throughput community sequencing data. Nature methods 7, 335-336.

Clayton, T. A., Baker, D., Lindon, J. C., Everett, J. R., and Nicholson, J. K. (2009). Pharmacometabonomic identification of a significant host-microbiome metabolic interaction affecting human drug metabolism. Proceedings of the National Academy of Sciences of the United States of America 106, 14728-14733.

Dallmann, R., Viola, A. U., Tarokh, L., Cajochen, C., and Brown, S. A. (2012). The human circadian metabolome. Proceedings of the National Academy of Sciences of the United States of America 109, 2625-2629.

Dennis, G., Jr., Sherman, B. T., Hosack, D. A., Yang, J., Gao, W., Lane, H. C., and Lempicki, R. A. (2003). DAVID: Database for Annotation, Visualization, and Integrated Discovery. Genome Biol 4, P3.

Eckel-Mahan, K. L., Patel, V. R., Mohney, R. P., Vignola, K. S., Baldi, P., and Sassone-Corsi, P. (2012). Coordination of the transcriptome and metabolome by the circadian clock. Proceedings of the National Academy of Sciences of the United States of America 109, 5541-5546.

Gury-BenAri, M., Thais s, C. A., Serafini, N., Winter, D. R., Giladi, A., Lara-Astiaso, D., Levy, M., Salame, T. M., Weiner, A., David, E., et al. (2016). The Spectrum and Regulatory Landscape of Intestinal Innate Lymphoid Cells Are Shaped by the Microbiome. Cell 166, 1231-1246 e1213.

Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589.

Hooper, L. V., Littman, D. R., and Macpherson, A. J. (2012). Interactions between the microbiota and the immune system. Science 336, 1268-1273.

Hughes, M.E., Hogenesch, J.B., and Kornacker, K. (2010). JTK_CYCLE: an efficient nonparametric algorithm for detecting rhythmic components in genome-scale data sets. Journal of biological rhythms 25, 372-380.

Jaitin, D. A., Kenigsberg, E., Keren-Shaul, H., Elefant, N., Paul, F., Zaretsky, I., Mildner, A., Cohen, N., Jung, S., Tanay, A., et al. (2014). Massively parallel single-cell RNA-seq for marker-free decomposition of tissues into cell types. Science 343, 776-779.

Johansson, M. E., Phillipson, M., Petersson, J., Velcich, A., Holm, L., and Hansson, G. C. (2008). The inner of the two Muc2 mucin-dependent mucus layers in colon is devoid of bacteria. Proceedings of the National Academy of Sciences of the United States of America 105, 15064-15069.

Johnson, B. P., Walisser, J. A., Liu, Y., Shen, A. L., McDearmon, E. L., Moran, S. M., McIntosh, B. E., Vollrath, A. L., Schook, A. C., Takahashi, J. S., et al. (2014). Hepatocyte circadian clock controls acetaminophen bioactivation through NADPH-cytochrome P450 oxidoreductase. Proceedings of the National Academy of Sciences of the United States of America 111, 18757-18762.

Kanehisa, M., and Goto, S. (2000). KEGG: kyoto encyclopedia of genes and genomes. Nucleic acids research 28, 27-30.

Kim, Y. C., and Lee, S. J. (1998). Temporal variation in hepatotoxicity and metabolism of acetaminophen in mice. Toxicology 128, 53-61.

Koike, N., Yoo, S. H., Huang, H. C., Kumar, V., Lee, C., Kim, T. K., and Takahashi, J. S. (2012). Transcriptional architecture and chromatin landscape of the core circadian clock in mammals. Science 338, 349-354.

Langmead, B., Trapnell, C., Pop, M., and Salzberg, S. L. (2009). Ultrafast and memory-efficient alignment of short DNA sequences to the human genome. Genome Biol 10, R25.

Leone, V., Gibbons, S. M., Martinez, K., Hutchison, A. L., Huang, E. Y., Cham, C. M., Pierre, J. F., Heneghan, A. F., Nadimpalli, A., Hubert, N., et al. (2015). Effects of diurnal variation of gut microbes and high-fat feeding on host circadian clock function and metabolism. Cell host & microbe 17, 681-689.

Levy, M., Thaiss, C. A., Zeevi, D., Dohnalova, L., Zilberman-Schapira, G., Mandi, J. A., David, E., Savidor, A., Korem, T., Herzig, Y., et al. (2015). Microbiota-Modulated Metabolites Shape the Intestinal Microenvironment by Regulating NLRP6 Inflammasome Signaling. Cell 163, 1428-1443.

Li, J., Jia, H., Cai, X., Zhong, H., Feng, Q., Sunagawa, S., Arumugam, M., Kultima, J. R., Prifti, E., Nielsen, T., et al. (2014). An integrated catalog of reference genes in the human gut microbiome. Nat Biotechnol 32, 834-841.

Liang, X., Bushman, F. D., and FitzGerald, G. A. (2015). Rhythmicity of the intestinal microbiota is regulated by gender and the host circadian clock. Proceedings of the National Academy of Sciences of the United States of America 112, 10479-10484.

Loonen, L. M., Stolte, E. H., Jaklofsky, M. T., Meijerink, M., Dekker, J., van Baarlen, P., and Wells, J. M. (2014). REG3gamma-deficient mice have altered mucus distribution and increased mucosal inflammatory responses to the microbiota and enteric pathogens in the ileum. Mucosal immunology 7, 939-947.

Marco-Sola, S., Sammeth, M., Guigo, R., and Ribeca, P. (2012). The GEM mapper: fast, accurate and versatile alignment by filtration. Nature methods 9, 1185-1188.

Masri, S., Papagiannakopoulos, T., Kinouchi, K., Liu, Y., Cervantes, M., Baldi, P., Jacks, T., and Sassone-Corsi, P. (2016). Lung Adenocarcinoma Distally Rewires Hepatic Circadian Homeostasis. Cell 165, 896-909.

Minami, Y., Kasukawa, T., Kakazu, Y., ligo, M., Sugimoto, M., Ikeda, S., Yasui, A., van der Horst, G. T., Soga, T., and Ueda, H. R. (2009). Measurement of internal body time by blood metabolomics. Proceedings of the National Academy of Sciences of the United States of America 106, 9890-9895.

Mukherji, A., Kobiita, A., Ye, T., and Chambon, P. (2013). Homeostasis in intestinal epithelium is orchestrated by the circadian clock and microbiota cues transduced by TLRs. Cell 153, 812-827.

Murakami, M., Tognini, P., Liu, Y., Eckel-Mahan, K. L., Baldi, P., and Sassone-Corsi, P. (2016). Gut microbiota directs PPARgamma-driven reprogramming of the liver circadian clock by nutritional challenge. EMBO Rep 17, 1292-1303.

Nakahata, Y., Kaluzova, M., Grimaldi, B., Sahar, S., Hirayama, J., Chen, D., Guarente, L. P., and Sassone-Corsi, P. (2008). The NAD+-dependent deacetylase SIRT1 modulates CLOCK-mediated chromatin remodeling and circadian control. Cell 134, 329-340.

Nakahata, Y., Sahar, S., Astarita, G., Kaluzova, M., and Sassone-Corsi, P. (2009). Circadian control of the NAD+ salvage pathway by CLOCK-SIRT1. Science 324, 654-657.

Neufeld-Cohen, A., Robles, M. S., Aviram, R., Manella, G., Adamovich, Y., Ladeuix, B., Nir, D., Rousso-Noori, L., Kuperman, Y., Golik, M., et al. (2016). Circadian control of oscillations in mitochondrial rate-limiting enzymes and nutrient utilization by PERIOD proteins. Proceedings of the National Academy of Sciences of the United States of America 113, E1673-1682.

Panda, S., Antoch, M. P., Miller, B. H., Su, A. I., Schook, A. B., Straume, M., Schultz, P. G., Kay, S. A., Takahashi, J. S., and Hogenesch, J. B. (2002). Coordinated transcription of key pathways in the mouse by the circadian clock. Cell 109, 307-320.

Panda, S., and Hogenesch, J. B. (2004). It's all in the timing: many clocks, many outputs. Journal of biological rhythms 19, 374-387.

Perelis, M., Marcheva, B., Ramsey, K. M., Schipma, M.J ., Hutchison, A. L., Taguchi, A., Peek, C. B., Hong, H., Huang, W., Omura, C., et al. (2015). Pancreatic beta cell enhancers regulate rhythmic transcription of genes controlling insulin secretion. Science 350, aac4250.

Ramsey, K. M., Yoshino, J., Brace, C. S., Abrassart, D., Kobayashi, Y., Marcheva, B., Hong, H. K., Chong, J. L., Buhr, E. D., Lee, C., et al. (2009). Circadian clock feedback cycle through NAMPT-mediated NAD+ biosynthesis. Science 324, 651-654.

Robertson, B. R., O'Rourke, J. L., Neilan, B. A., Vandamme, P., On, S. L., Fox, J. G., and Lee, A. (2005). Mucispirillum schaedleri gen. nov., sp. nov., a spiral-shaped bacterium colonizing the mucus layer of the gastrointestinal tract of laboratory rodents. International journal of systematic and evolutionary microbiology 55, 1199-1204.

Thaiss, C. A., Levy, M., and Elinav, E. (2015a). Chronobiomics: The Biological Clock as a New Principle in Host-Microbial Interactions. PLoS pathogens 11, e1005113.

Thaiss, C. A., Zeevi, D., Levy, M., Segal, E., and Elinav, E. (2015b). A day in the life of the meta-organism: diurnal rhythms of the intestinal microbiome and its host. Gut Microbes 6, 137-142.

Thaiss, C. A., Zeevi, D., Levy, M., Zilberman-Schapira, G., Suez, J., Tengeler, A. C., Abramson, L., Katz, M. N., Korem, T., Zmora, N., et al. (2014). Transkingdom control of microbiota diurnal oscillations promotes metabolic homeostasis. Cell 159, 514-529.

Trapnell, C., Pachter, L., and Salzberg, S. L. (2009). TopHat: discovering splice junctions with RNA-Seq. Bioinformatics 25, 1105-1111.

Vaishnava, S., Yamamoto, M., Severson, K. M., Ruhn, K. A., Yu, X., Koren, O., Ley, R., Wakeland, E. K., and Hooper, L. V. (2011). The antibacterial lectin RegIIIgamma promotes the spatial segregation of microbiota and host in the intestine. Science 334, 255-258.

Voigt, R. M., Forsyth, C. B., Green, S. J., Mutlu, E., Engen, P., Vitaterna, M. H., Turek, F. W., and Keshavarzian, A. (2014). Circadian disorganization alters intestinal microbiota. PloS one 9, e97500.

Voigt, R. M., Summa, K. C., Forsyth, C. B., Green, S. J., Engen, P., Naqib, A., Vitaterna, M. H., Turek, F. W., and Keshavarzian, A. (2016). The Circadian Clock Mutation Promotes Intestinal Dysbiosis. Alcohol Clin Exp Res 40, 335-347.

Vollmers, C., Schmitz, R. J., Nathanson, J., Yeo, G., Ecker, J. R., and Panda, S. (2012). Circadian oscillations of protein-coding and regulatory RNAs in a highly dynamic mammalian liver epigenome. Cell metabolism 16, 833-845.

Wlodarska, M., Thaiss, C. A., Nowarski, R., Henao-Mejia, J., Zhang, J. P., Brown, E. M., Frankel, G., Levy, M., Katz, M. N., Philbrick, W. M., et al. (2014). NLRP6 inflammasome orchestrates the colonic host-microbial interface by regulating goblet cell mucus secretion. Cell 156, 1045-1059.

Zarrinpar, A., Chaix, A., Yooseph, S., and Panda, S. (2014). Diet and feeding pattern affect the diurnal dynamics of the gut microbiome. Cell metabolism 20, 1006-1017.

Zhang, R., Lahens, N. F., Ballance, H. I., Hughes, M. E., and Hogenesch, J. B. (2014). A circadian gene expression atlas in mammals: implications for biology and medicine. Proceedings of the National Academy of Sciences of the United States of America 111, 16219-16224.

Zwighaft, Z., Aviram, R., Shalev, M., Rousso-Noori, L., Kraut-Cohen, J., Golik, M., Brandis, A., Reinke, H., Aharoni, A., Kahana, C., et al. (2015). Circadian Clock Control by Polyamine Levels through a Mechanism that Declines with Age. Cell metabolism 22, 874-885.

1-2. (canceled)

3. A method of reducing the liver toxicity of a liver-damaging agent in a subject comprising administering to the subject:
(i) the liver-damaging agent; and
(ii) an agent which alters the circadian rhythmicity of microbes of the gut microbiome of the subject, thereby reducing the liver toxicity of the liver-damaging agent.

4. A method of treating a liver disease in a subject in need thereof comprising administering to the subject:
(i) a therapeutically effective amount of an agent which is therapeutic for the liver disease; and
(ii) an agent which alters the circadian rhythmicity of microbes of the gut microbiome of the subject, thereby treating the liver disease.

5. A method of selecting a dose or treatment regimen of a pharmaceutical agent for a subject comprising:
(a) analyzing the circadian rhythmicity of at least one component of the gut microbiome of the subject; and
(b) selecting the dose or treatment regimen according to said circadian rhythmicity.

6. The method of claim 3, wherein said agent which alters the circadian rhythmicity of microbes of the gut microbiome is an antibiotic.

7. The method of claim 6, wherein said antibiotic is selected from the group consisting of Ampicillin, Neomycin, Vancomycin and Mettronidazole.

8. The method of claim 3, wherein said agent which alters the circadian rhythmicity of microbes of the gut microbiome is a probiotic.

9. The method of claim 8, wherein said probiotic comprises a polyamine producing bacteria.

10. The method of claim 3, wherein said agent comprises a food which is rich in polyamines.

11. The method of claim 3, wherein said agent comprises a diet which is a polyamine deficient diet or a fat rich diet.

12. The method of claim 3, wherein said agent is a metabolite whose abundance follows a circadian rhythmicity in the gut microbiome.

13. The method of claim 5, wherein said at least one component is a metabolite.

14. The method of claim 12, wherein said metabolite is a polyamine.

15. The method of claim 14, wherein said polyamine is selected from the group consisting of putrescine, cadaverine, spermidine and spermine.

16. The method of claim 3, wherein said agent is a fecal microbiome transplant derived from a healthy donor.

17. The method of claim 3, wherein the liver-damaging agent is a pharmaceutical agent.

18. The method of claim 17, wherein the pharmaceutical agent is acetaminophen.

19. (canceled)

20. The method of claim 3, wherein the liver-damaging agent is administered in a higher unit dosage than the pharmaceutically acceptable unit dosage thereof.

21. The method of claim 4, wherein said therapeutically effective amount of said agent is higher than the pharmaceutically acceptable unit dosage of said agent.

22. The method of claim 3, wherein said agent which alters the circadian rhythmicity of the gut microbiome is selected by analyzing the gut microbiome of the subject prior to the administering.


SEQUENCE LISTING (13 sequences)

SEQ ID NO: 1 - DNA, 19 bp, Artificial sequence
  Single strand DNA oligonucleotide
  misc_feature (2)..(2): n is a, c, g, or t
  misc_feature (18)..(18): n is a, c, g, or t
cnacgcgaagaaccttanc

SEQ ID NO: 2 - DNA, 19 bp, Artificial sequence
  Single strand DNA oligonucleotide
atacgcgargaaccttacc

SEQ ID NO: 3 - DNA, 19 bp, Artificial sequence
  Single strand DNA oligonucleotide
  misc_feature (9)..(9): n is a, c, g, or t
ctaaccgangaacctyacc

SEQ ID NO: 4 - DNA, 19 bp, Artificial sequence
  Single strand DNA oligonucleotide
caacgcgmaraaccttacc

SEQ ID NO: 5 - DNA, 19 bp, Artificial sequence
  Single strand DNA oligonucleotide
  misc_feature (14)..(14): n is a, c, g, or t
cgacrrccatgcancacct

SEQ ID NO: 6 - DNA, 18 bp, Artificial sequence
  16S probe oligonucleotide sequence
  misc_feature (1)..(1): 5′ CAL flour Red 610
  misc_feature (18)..(18): 3′ BHQ-2
gctgcctcccgtaggagt

SEQ ID NO: 7 - DNA, 28 bp, Artificial sequence
  barcoded PCR primer
  misc_feature (1)..(8): barcode sequence
nnnnnnnnagagtttgatcctggctcag

SEQ ID NO: 8 - DNA, 19 bp, Artificial sequence
  Single strand DNA oligonucleotide
tgctgcctcccgtaggagt

SEQ ID NO: 9 - DNA, 64 bp, Artificial sequence
  Chromatin first indexing adapter single strand DNA
  misc_feature (1)..(1): 5′ C3 spacer
  misc_feature (56)..(63): barcoding sequence
  misc_feature (56)..(63): n is a, c, g, or t
cgattgaggccggtaatacgactcactataggggcgacgtgtgctcttccgatctnnnnnnnnt

SEQ ID NO: 10 - DNA, 31 bp, Artificial sequence
  Ligation adapter
  misc_feature (1)..(1): 5′  phosphate group
  misc_feature (1)..(1): n is a, c, g, or t
  misc_feature (2)..(9): n is a, c, g, or t
  misc_feature (31)..(31): C3 spacer
nnnnnnnnnagatcggaagagcgtcgtgtag

SEQ ID NO: 11 - DNA, 22 bp, Artificial sequence
  Single strand DNA oligonucleotide
tctagccttctcgcagcacatc

SEQ ID NO: 12 - DNA, 42 bp, Artificial sequence
  Single strand DNA oligonucleotide
aatgatacggcgaccaccgagatctacactctttccctacac

SEQ ID NO: 13 - DNA, 41 bp, Artificial sequence
  Single strand DNA oligonucleotide
caagcagaagacggcatacgagatgtgactggagttcagac